(19) United States
(12) Patent Application Publication
(10) Pub. No.: US 2018/0252705 A1
(43) Pub. Date: Sep. 6, 2018

(54) Myeloid Progenitor Cells in Kidney Disease

(71) Applicants: Rush University Medical Center, Chicago, IL (US); Massachusetts General Hospital, Boston, MA (US)

(72) Inventors: Jochen Reiser, Chicago, IL (US); Eunsil Hahm, Morton, IL (US); Sanja Sever, Brookline, MA (US)

(73) Assignees: Rush University Medical Center, Chicago, IL (US); Massachusetts General Hospital, Boston, MA (US)

(21) Appl. No.: 15/560,926

(22) PCT Filed: Mar. 25, 2016
(86) PCT No.: PCT/US16/24229

§ 371 Date: Sep. 22, 2017

Related U.S. Application Data
(60) Provisional application No. 62/139,454, filed on Mar. 27, 2015.

(51) Int. Cl. G01N 33/50; A61P 13/12; A61K 31/711; A61K 31/7115

(52) U.S. Cl. CPC G01N 33/5088; A61P 13/12; A61K 2123/00; G01N 33/5044; A61K 31/7115; A61K 31/711

(57) ABSTRACT

Methods of treating kidney diseases or disorders and methods of identifying agents for treatment of kidney diseases or disorders are provided. The method includes administering to a subject in need thereof, an effective amount of an agent which inhibits myeloid progenitor cells in the subject from producing soluble urokinase receptor (suPAR). The method includes administering the agent to a patient, where the patient, has an increased number of Gr-1 low cells relative to a control.

### RELATED APPLICATIONS

This application claims the benefit of U.S. Provisional Application No. 62/139,454, filed Mar. 27, 2015, which is incorporated by reference herein in its entirety.

### BACKGROUND

### 1. Technical Field

Methods of treating kidney diseases or disorders and methods of identifying agents for treatment of kidney diseases or disorders are provided.

### 2. Background

Focal segmental glomerulosclerosis (FSGS) is a common primary glomerular kidney disorder characterized clinically by proteinuria and morphologically by segmental sclerosis in some glomeruli (Winn et al., 2005; D'Agati et al.). Primary FSGS eventually leads to kidney failure, necessitating dialysis or kidney transplantation (Cravedi et al.). However, the high incidence of FSGS recurrence in both children and adults following transplant have led to the presumption that a T-cell mediated circulating factor has been considered as a pathogenic cause known as the Shalhoub hypothesis (Fogo; Gallon et al.; Shalhoub, 1974).

We previously reported that soluble urokinase plasminogen activator receptor (suPAR) is one of such circulating factors that may cause FSGS, and demonstrated that suPAR binds to and activates β3 integrin on the podocyte membrane, leading to podocyte foot process effacement and disrupted glomerular barrier function with proteinuria (Wei et al., 2008; Wei et al.). Circulating suPAR is generated by release from the membrane-bound form, urokinase plasminogen activator receptor (uPAR), a glycosylphosphatidylinositol (GPI)-anchored three domain (DI, DII, and DIII) protein (Thuno et al., 2009; Blasi et al., 2002). SuPAR exists in multiple forms due to alternative splicing, protein glycosylation, and enzymatic cleavage of the mature protein (Smith et al.). The biochemical composition of suPAR is a critical determinant in disease initiation and severity, as not all individuals with high plasma suPAR develop FSGS.

Despite convincing experimental and clinical evidence that suPAR may be the circulating factor causing human FSGS, the cellular sources of elevated suPAR remain unknown. Moreover, whether the originating cells of the pathogenic suPAR are sufficient to cause the disease in healthy mice has not been tested.

### BRIEF SUMMARY

A method of treating kidney diseases or disorders is provided. The method includes administering to a subject in need thereof, an effective amount of an agent which inhibits myeloid progenitor cells in the subject from producing soluble urokinase receptor (suPAR).

A method of identifying an agent for treatment of kidney diseases or disorders is provided. The method includes administering the agent to a murine host where the murine host has an increased number of Gr-1^{lo} cells relative to a control. The method also includes determining the effect of the agent on an indicator of kidney disease and identifying the agent as useful in treating kidney disease when the agent ameliorates the indicator of kidney disease in the murine host relative to the control.

### BRIEF DESCRIPTION OF THE DRAWINGS

FIGS. 1A-1I. Bone marrow myeloid cells (BMCs) are required for suPAR-mediated proteinuria.

FIG. 1A: Flow cytometric analysis of uPAR expression in hematopoietic cells. PBLs and BMCs were isolated from control (PBS-treated) and proteinuric (LPS-treated) mice and labeled with fluorescence-conjugated antibodies specific for uPAR and myeloid specific differentiation antigen, Gr-1 (n=4-5 mice per group from two independent experiments). The left panel shows representative dot plots of the two-color staining (uPAR/Gr-1) in the gated myeloid cell population (using forward and side scatter, data not shown). The overlay histograms (right panel) display the expression profiles of uPAR on the gated myeloid cells from PB (upper) and BM (lower). Background fluorescence (gray line) was determined with an irrelevant isotype-matched antibody. Blue line, PBS; red line, LPS.

FIGS. 1B-1D: NSG mice were injected with either PBS or LPS, then urine and blood were collected 24 hours after LPS administration (n=4 per group from two independent experiments). FIG. 1B: Urinary albumin and creatinine were measured by mouse albumin ELISA and creatinine assay, respectively. Then, albumin-to-creatinine ratio (ACR) was calculated and used as a parameter to determine proteinuria. FIG. 1C: Mouse serum suPAR levels were evaluated by ELISA method. FIG. 1D: Flow cytometry analysis of uPAR expression on BM myeloid cells. Data are the same as presented in FIG. 1A.

FIG. 1E: Schematic experimental design to study the role of BM myeloid cells in proteinuria development and suPAR production.

FIGS. 1F and 1G: BALB/c mice were irradiated or not irradiated, then injected or not injected with freshly isolated BMCs. Urine and blood were collected 24 hours after LPS administration (n=5-8 per group from two independent experiments). FIG. 1F: ACR. FIG. 1G: plasma suPAR levels.

FIGS. 1H and 1I: WT B6 mice were irradiated or not irradiated, then injected or not injected with BMCs of WT or Plaur^{-/-} (KO, B6 background) mice. Then, urine and blood were collected 24 hours after LPS administration (n=9-11 per group from four independent experiments). FIG. 1H: ACR. FIG. 1I: Plasma suPAR levels. Data are shown as mean±SD (FIGS. 1B and 1C) or ±SEM (FIGS. 1F-1I). Student's t-test, *P<0.05, **P<0.01, ***P<0.001, N.S, not significant.

FIGS. 2A-2I. BM myeloid progenitor cells are responsible for suPAR-mediated proteinuria.

FIGS. 2A and 2B: Flow cytometry analysis of Gr-1 expression in BMCs from mice treated as in FIG. 1A. FIG. 2A: Representative dot plots showing percentages of Gr-1^{high}, Gr-1^{low}, and Gr-1^{neg} cells in the gated BM myeloid cell population (using forward and side scatter, data not shown). FIG. 2B: The bar graph shows percentages of Gr-1^{high} (open bar), Gr-1^{low} (filled bar) cells.

FIG. 2C: ACR levels of neutropenic (anti-Ly6G antibody-injected) and control (isotype antibody-injected) mice following LPS (or PBS) administration (n=5-7 per group from two independent experiments).

FIG. 2D: ACR levels of G-CSF (or PBS)-treated mice prior to LPS (or PBS) stimulation (n=6-7 per group from two independent experiments).

FIG. 2E: Plasma suPAR levels in mice treated as in FIG. 2D.

FIG. 2F: ACR levels of plerixafor (or PBS)-treated mice prior to LPS administration (n=5-6 per group from two independent experiments).

FIG. 2G-1: WT mice were injected with either LPS or PBS. After 24 hours, the BMCs were isolated and labeled with fluorescence-conjugated antibodies specific for uPAR, Sca-1, and Gr-1 then, analyzed by flow cytometry (n=4 per group from two independent experiments). FIG. 2G: From the dot plots of uPAR/Sca-1 (left panel), uPAR⁺Sca-1⁻ (pink) and uPAR⁺Sca-1⁺ (green) cells were gated and shown in the dot plots of Gr-1/Sca-1 (right panel) to identify Sca-1⁺Gr-1^{low} BMCs as candidate for uPAR producing cells in LPS-proteinuric mice.

FIG. 2H: The bar graph shows percentages of uPAR⁺Sca-1⁻Gr-1⁺ (pink bar) and uPAR⁺Sca-1⁺Gr-1⁺ (green bar) cells in total BMCs from PBS (open bar) or LPS (filled bar) injected mice. FIG. 2I: The graph shows averaged mean fluorescent intensity (MFI) of Gr-1 in uPAR-positive BMCs from PBS or LPS injected mice. Data are shown as mean±SD (FIGS. 2B, 2H, and 2I) or ±SEM (FIGS. 2C-2F). Student's t-test, *P<0.05, **P<0.01, ***P<0.001, N.S, not significant.

FIGS. 3A-3L. Transfer of mouse or human FSGS to mouse.

FIGS. 3A-3C: WT and Plaur^{-/-} (KO) mice were challenged with LPS for 24 hours, and then BMCs were isolated from those mice and transferred into NSG mice. ACR and suPAR levels were monitored in a time course (n=6 per group from two independent experiments). FIG. 3A: Schematic experimental design. FIG. 3B: ACR. FIG. 3C: Serum suPAR levels.

FIGS. 3D-3F: To test whether Sca-1⁺ cells are involved in suPAR-mediated proteinuria, NSG mice were adoptively transferred with either whole or Sca-1⁺ cell-depleted (Sca-1^{neg}) BMCs of LPS-challenged WT mice. ACR and suPAR levels were monitored in a time course (n=8-9 per group from three independent experiments). FIG. 3E: ACR. FIG. 3F: Serum suPAR levels.

FIG. 3G: To test whether BMCs of mice having proteinuria by kidney podocyte injury are capable of causing proteinuria, NSG mice were adoptively transferred with BMCs of proteinuric double transgenic (dTG; NEF-rtTAxRac1) and normal control (Rac1) mice. Double transgenic (dTG; NEF-rtTAxRac1) mice were fed with DOX to induce proteinuria. Single transgenic (Rac1) mice were used as a control. (n=3-6 per group from two independent experiments). As a positive control, LPS-challenged BMCs were also transferred into NSG mice. ACR was measured from the urine samples collected from the recipient NSG mice in a time course.

FIGS. 3H-3L: To generate xenograft mice, human PBMCs were isolated from 2 different patients with FSGS and healthy donors, then injected into NSG mice on day 0. The engrafted mice were monitored overtime for the development of FSGS-like phenotypes by monitoring in the blood and urine; proteinuria, high suPAR levels. Urine, blood, and kidney were harvested from the humanized NSG mice on day 90. FIG. 3H: Schematic experimental design. FIG. 3I: Engraftment rates of human cells were determined by the percentage of human CD45⁺ cells in blood and BM of the NSG mice at 12 weeks post engraftment. FIG. 3J: Photographs of representative humanized mice on 12 weeks post-engraftment of human PBMCs. None of the mice engrafted with healthy PBMCs showed proteinuria. n=2 per group. FIG. 3K: ACR. FIG. 3L: Plasma suPAR levels. Data are shown as mean±SD (FIGS. 3G, 3I, 3K, and 3L) or ±SEM (FIGS. 3B-3F). Student's t-test, *P<0.05, N.S, not significant.

FIGS. 4A-4G. FSGS CD34⁺ cells trigger expansion of Gr-1^{low} MPCs, leading to disease development.

FIGS. 4A-4F: The xenograft mice were generated by injecting NSG mice with (i) non-depleted (whole) or (ii) CD34⁺ cell depleted (CD34⁻) PBMCs from healthy donors, (iii) whole or (iv) CD34⁻ PBMCs from patients with FSGS on day 0. The mice were monitored overtime for the development of FSGS-like phenotypes by monitoring in the blood and urine; proteinuria and high suPAR levels. Urine, blood, and kidney were harvested from the NSG mice on 10 weeks post-engraftment. FIG. 4A: Photographs of representative humanized mice (n=2-5 per group). FIG. 4B: ACR. FIG. 4C: Plasma suPAR levels. FIG. 4D: Flow cytometry analysis of Gr-1 expression on BM myeloid cells of the humanized mice. Bar graph shows the proportions of Gr-1^{high} (open bar) and Gr-1^{low} (filled bar) populations. FIG. 4E: Flow cytometry analysis of uPAR expression on BM myeloid cells of the humanized mice. Bar graph shows MFI values of uPAR staining. MFI from BM myeloid cells of NSG mice engrafted with healthy whole PBMCs was set as 100%. FIG. 4F: Sections of formalin-fixed kidney glomeruli from the humanized mice were stained with H&E and with PAS. Scale bars, 50 μm. Transmission and scanning electron microscope (TEM, 10,000× and SEM, 15,000×) analysis of kidney glomeruli of the humanized mice. TEM images displaying podocyte foot processes were enlarged and highlighted. SEM images show a podocyte cell body, primary processes and interdigitating foot processes. Scale bars, 2 μm.

FIG. 4G: Hypothetical model depicting that pathogenic myeloid progenitor cells control kidney disease process via production of pathogenic suPAR.

FIGS. 5A-5B. BM myeloid cells are responsible for development of proteinuria.

FIG. 5A: Urine samples were collected from the mice given i) PBS, ii) LPS, iii) irradiation+LPS, and iv) irradiation+BMC+LPS 24 hours after LPS (or PBS) administration. One microliter of mouse urine resolved on a 10% SDS-PAGE gel. Urinary proteins are stained with Gelcode Blue. Bovine serum albumin (BSA) was used as the standard. FIG. 5B: Bar graph showing albumin/creatinine ratio (ACR) in each group. The intensities of bands were measured by densitometric analysis. Albumin levels from urine were calculated using a BSA standard curve (n=2).

FIGS. 6A-6E. Increased myelopoiesis facilitated proteinuria.

FIGS. 6A and 6B: PBLs and BMCs were isolated from neutropenic (anti-Ly6G antibody-injected) and control (isotype antibody-injected) mice. FIG. 6A: Representative flow cytometry plots of PBLs and BMCs from control IgG or anti-Ly6G antibody injected mice. Myeloid cell populations (red circle) were gated based on their forward scatter (FSC) and side scatter (SSC) properties (n=4).

FIG. 6B: The bar graph shows quantitative analysis of myeloid cell numbers measured by flow cytometry (n=4).

FIGS. 6C-6E: BALB/c mice were treated with G-CSF (or PBS) for two consecutive days prior to LPS (or PBS) stimulation. Blood and bones (femurs and tibias) were collected 24 hours after LPS administration. FIG. 6C: The bar graph shows quantitative analysis of white blood cell (WBC) counts determined using a Hemavet (n=3). FIG. 6D: PBLs and BMCs were isolated and labeled with fluorescence-conjugated antibodies specific for Gr-1. Representative dot plots showing percentages of Gr-1^{high}, Gr-1^{low}, and Gr-1^{neg} cells in the gated myeloid cell population (using forward and side scatter). FIG. 6E: The bar graph shows quantitative analysis. Data are shown as mean±SD. Student's t-test, **P<0.01, ***P<0.001.

FIGS. 7A-7D. Sca-1⁺ BMCs are involved in suPAR-mediated proteinuria.

FIG. 7A: Whole BMCs were isolated from WT and KO (Plaur^{-/-}) mice treated with either LPS or PBS. The BMCs were labeled with fluorescence-conjugated antibodies specific for Sca-1 and c-kit, and analyzed by flow cytometry. The percentage of BMCs positive for Sca-1 and/or c-kit was presented as bar graph (n=4 per group from two independent experiments).

FIG. 7B: Confirmation of Sca-1⁺ cell depletion using flow cytometric analysis of intact, Sca-1⁺ cell-depleted BMCs. BMCs were isolated from WT mice treated with LPS. Sca-1⁺ cells were removed from whole BMCs by magnetic separation. Cells were stained with FITC conjugated anti-Sca-1 or isotype control antibodies. Representative histograms display the expression profiles of Sca-1 (n=2). Blue dashed line indicates the cutoff for a positive Sca-1 signal. Sca-1-positive cells are encircled in red.

FIG. 7C: Schematic experimental design to test whether Sca-1⁺ cells are involved in suPAR-mediated proteinuria. BMCs were isolated from LPS-challenged WT mice. Sca-1⁺ cell-depleted (Sca-1^{neg}) BMCs were prepared using a depletion kit. The NSG mice were received either whole or Sca-1^{neg} BMCs. ACR and suPAR levels were monitored in a time course (n=8-9 per group from three independent experiments).

FIG. 7D: Schematic experimental design to test whether BMCs of mice having proteinuria by kidney podocyte injury are capable of causing proteinuria. Double transgenic (dTG; NEF-rtTAxRac1) mice were fed with DOX to induce proteinuria. Single transgenic (Rac1) mice were used as a control. BMCs were isolated from proteinuric dTG and normal control (Rac1) mice then, transferred into NSG mice (n=3-6 per group from two independent experiments). As a positive control, LPS-challenged BMCs were also transferred into NSG mice. Data are shown as mean±SD. Student's t-test, **P<0.01, ***P<0.001.

FIGS. 8A-8D. Kidney functions in the xenograft mice.

FIGS. 8A-8D: Non-depleted (whole) or CD34⁺ cell depleted (CD34⁻) PBMCs from patients with FSGS and normal individuals were transferred into NSG mice. Blood and kidney were harvested from the NSG mice on 10 weeks post-engraftment (n=2-5 per group). FIG. 8A: Urinary suPAR levels. FIG. 8B: Blood urea nitrogen (BUN), as a marker for kidney function, was measured in serum samples from the humanized mice using a colorimetric-based assay kit (BioAssay Systems). FIG. 8C: Kidney weights. FIG. 8D: Representative SEM images of whole glomeruli of the humanized mice. Scale bar, 10 μm. Data are shown as mean±SD.

FIGS. 9A-9J. BM myeloid cells are required for suPAR-mediated proteinuria. (9A-9C) BM chimeric mice were made by irradiation with a dose of 9.5 Gy and reconstitution via retro-orbital injection with 1×10⁷ donor BM cells. Mice were administered antibiotic-treated water and used for experiments at 6 weeks after BMT. The BM chimeric (WT→KO and KO→KO) mice were injected with LPS. Blood and urine samples were collected at 24 hours after LPS injection. (9A) serum suPAR levels. (9B) urinary suPAR levels. (9C) Urinary albumin and creatinine were measured by mouse albumin ELISA and creatinine assay, respectively. Then, albumin-to-creatinine ratio (ACR) was calculated and used as a parameter to determine proteinuria. Data are shown as mean±SD. Student t-test, *P<0.05, ***P<0.001. (9D-9F) NSG mice were injected with either PBS or LPS, then urine and blood were collected 24 hours after LPS administration (n=4 per group from two independent experiments). (9D) serum suPAR levels. (9E) urinary suPAR levels. (9F) proteinuria. Data are shown as mean±SD. Student t-test, *P<0.05. (9G) Flow cytometric analysis of uPAR expression in hematopoietic cells. PBLs and BMCs were isolated from control (PBS-treated) and proteinuric (LPS-treated) mice and labeled with fluorescence-conjugated antibodies specific for uPAR and myeloid specific differentiation antigen, Gr-1 (n=4-5 mice per group from two independent experiments). The overlay histograms display the expression profiles of uPAR on the gated myeloid cells (using forward and side scatter, data not shown) from PB (up) and BM (down). Background fluorescence (gray line) was determined with an irrelevant isotype-matched antibody. Blue line, PBS; red line, LPS. (9H-9J) BALB/c mice were irradiated or not irradiated, then injected or not injected with freshly isolated BMCs. Urine and blood were collected 24 hours after LPS administration (n=5-8 per group from two independent experiments). (9H) Schematic experimental design. (9I) proteinuria. (9J) plasma suPAR levels. Data are shown as mean±SEM. One-way ANOVA, followed up by Tukey's multiple comparison test, *P<0.05, **P<0.01, ***P<0.001.

FIGS. 10A-10G. Expansion of Gr-1^{lo} BM cells are involved in suPAR-associated proteinuria. (10A and 10B) G-CSFR deficient (KO) and WT mice were injected with LPS or PBS, 24 hours later, Gr-1 expression on BM cells and proteinuria were evaluated (n=5-7 mice per group from two independent experiments). (10A) The bar graph shows percentages of Gr-1^{lo} cells in BM. (B) proteinuria. Data are shown as mean±SEM. One-way ANOVA, Turkey's multiple comparison test, **P<0.01, ***P<0.001, NS, not significant. (10C-F) Examination of proteinuria, suPAR levels, and Gr-1^{lo} BM myeloid cells in LPS model and Pod-Rac1, a genetic model of podocyte injury. (10C) proteinuria. (10D) serum suPAR. (10E) urinary suPAR. (10F) percentages of Gr-1^{lo} cells in BM. Data are shown as mean±SEM. Student t-test, **P<0.01, ***P<0.001, NS, not significant. (10G) Examination of proteinuria, suPAR levels, and Gr-1^{lo} BM myeloid cells in 3 different animal models of proteinuria; i) Albumin TGF β₁ transgenic (TGF β₁ Tg) mice, ii) nephrotoxic serum (NTS) nephritis, iii) BTBR ob/ob diabetic nephropathy (DN) models. Data are shown as mean±SEM. Student t-test, *P<0.05, **P<0.01, ***P<0.001, NS, not significant.

FIGS. 11A-11I. BM myeloid progenitor cells have an ability to transfer disease. (11A) Donor NOD-scid IL2rγ^{null} (NSG) mice were challenged with LPS or PBS for 24 hours, and then bone marrow cells (BMCs) were isolated from those mice and transferred into unchallenged recipient NSG mice (n=4-7 per group from two independent experiments). Urine samples were collected from the recipient mice before (0) and 6, 12, and 24 hour following BMC transfer. Proteinuria was evaluated in recipient NSG mice. Data are shown as mean±SEM. Student's t-test, *P<0.05. (11B) Adoptive transfer of BM cells from 2 different proteinuric mouse models (LPS and Pod-Rac1). To induce proteinuria in donor mice, i) WT C57BL/6 mice were injected with LPS or PBS, ii) Pod-Rac1 double transgenic (dTG; NEF-rtTAxRac1) or single transgenic (Rac1) mice were fed with doxycycline (DOX) for 12 days. Proteinuria was evaluated in recipient NSG mice 12 hours following BM cell transfer (n=5 per group). Data are shown as mean±SEM. One-way ANOVA, Turkey's multiple comparison test, **P<0.01, N.S, not significant. (11C) Representative dot plots of the triple-color staining (uPAR/Sca-1/Gr-1) in whole BM cells (red). BM cells were isolated from control (PBS-treated) and proteinuric (LPS-treated) mice and labeled with fluorescence-conjugated antibodies specific for uPAR, Gr-1, and Sca-1. uPAR+ cells (blue) were gated and shown in these dot plots. (11D) Quantitation for uPAR⁺Sca-1^{lo} Gr-1^{lo} cells shown in C. Data are shown as mean±SEM. Student's t-test, ***P<0.001. (11E and 11F) The BM cells were isolated from uPAR wild type mice, and treated with PBS or various concentrations of LPS (0.1, 1, and 10 μg/ml). Following 24-hour-incubation at 37 C in a CO₂ incubator (5%), the cells and culture supernatants were collected. (11E) The BM cells were stained for uPAR, Sca-1, and Gr-1. The in vitro induction of uPAR⁺Sca-1^{lo} Gr-1^{lo} cells in total BM cells was determined by triple color-flow cytometric analysis. (11F) The suPAR levels in the culture medium were measured by suPAR ELISA. Data are shown as mean±SEM. One-way ANOVA, Turkey's multiple comparison test, *P<0.05, **P<0.01, ***P<0.001. (11G-I) To test whether Sca-1⁺ cells are involved in suPAR-mediated proteinuria, NSG mice were adoptively transferred with either whole or Sca-1⁺ cell-depleted (Sca-10) BMCs of LPS-challenged WT mice. Proteinuria and suPAR levels were monitored in a time course (n=8-9 per group from three independent experiments). (11G) proteinuria. (11H) serum suPAR. (I) urinary suPAR. Data are shown as mean±SEM. Student's t-test, *P<0.05.

FIGS. 12A-12G. Engraftment of hFSGS CD34⁺ PBMCs developed suPAR-mediated proteinuria and elevated Gr-1^{lo} BM cells. (12A-F) A xenograft model of FSGS. (12A) Schematic experimental design. To generate xenograft mice, human PBMCs were isolated from patients with recurrent FSGS and healthy donors, then non-depleted (whole) or CD34⁺ cell depleted (CD34^{Δ}) PBMCs were transferred into recipient NSG mice on day 0. The engrafted mice were monitored overtime for the development of FSGS-like phenotypes by monitoring in the blood and urine; proteinuria and high suPAR levels. Urine, blood, and kidney were harvested from the NSG mice on 10-12 weeks post-engraftment. (n=4-5 per group from two independent experiments). (12B) proteinuria. (12C) plasma suPAR. (12D) urinary suPAR. (12E) percentages of Gr-1^{lo} cells in BM. Data are shown as mean±SEM. One-way ANOVA, followed up by Tukey's multiple comparison test, *P<0.05, **P<0.01, ***P<0.001. (12F) Transmission and scanning electron microscope (TEM, 10,000× and SEM, 15,000×) analysis of kidney glomeruli of the humanized mice. TEM images displaying podocyte foot processes were enlarged and highlighted. SEM images show a podocyte cell body, primary processes and interdigitating foot processes. Scale bars, 2 μm. (12G) Model depicting a role for Gr-1^{lo} BM myeloid cells in suPAR-driven podocyte injury/proteinuria. Systemic immunological proteinuric models—LPS, hFSGS xenograft, TGFβ1 transgenic (fibrosing nephrotic syndrome), NTS (serum nephritis), and BTBR ob/ob (diabetic nephropathy)—converge at the expansion of Gr-1^{lo} cells in BM and high blood suPAR levels.

FIGS. 13A-13D. Long-term exposure of suPAR resulted in podocyte injury and proteinuria in suPAR transgenic mice. (13A) suPAR transgenic mouse (suPAR-Tg) model was created that drives mouse full-length suPAR (corresponding to NP_035243, DIDIIDIII without GPI anchor) expression from adipocytes with consequent release into circulation. To stimulate suPAR production, regular rodent diet was replaced by high fat food when the mice were at least 2 months old. Two months after switching to high fat diet, blood and urine samples were collected from suPAR transgenic mice (suPAR-Tg, n=6) and their littermate controls (control, n=17). (13B) Plasma level of suPAR. (13C) Proteinuria development. (13D) TEM images (10,000×) displaying podocyte foot processes were enlarged and highlighted. Scale bars, 1 μm. Data are shown as mean±SD. Student's t-test, *P<0.05, ***<0.001.

FIG. 14. Engraftment rate of donor cells in BM chimeric mice. Irradiated uPAR deficient mice (uPAR KO, CD45.2) were received either uPAR WT (B6.SJL, CD45.1) or uPAR KO (CD45.2) BM cells. At 6 weeks after BM transplantation, engraftment of the donor cells was evaluated by flow cytometric analysis of peripheral blood leukocytes stained with fluorescence-conjugated antibodies against CD45.1 (uPAR WT) and CD45.2 (uPAR KO).

FIGS. 15A-15B. BM myeloid cells are responsible for development of proteinuria. (15A) Urine samples were collected from the mice given i) PBS, ii) LPS, iii) irradiation+LPS, and iv) irradiation+BMC+LPS 24 hours after LPS (or PBS) administration. One microliter of mouse urine resolved on a 10% SDS-PAGE gel. Urinary proteins are stained with Gelcode Blue. Bovine serum albumin (BSA) was used as the standard. (15B) Bar graph showing albumin/creatinine ratio (ACR) in each group. The intensities of bands were measured by densitometric analysis. Albumin levels from urine were calculated using a BSA standard curve (n=2).

FIGS. 16A-16B. LPS stimulation increased in the percentage of Gr-1^{lo} cells in BM. BM cells were isolated from control (PBS-treated) and proteinuric (LPS-treated) mice and labeled with fluorescence-conjugated antibodies specific for Gr-1 (n=4-5 mice per group from two independent experiments). (16A) Representative dot plots showing percentages of Gr-1^{high}, Gr-1^{low}, and Gr-1^{neg} cells in the gated BM myeloid cell population (using forward and side scatter, data not shown). (16B) The bar graph shows percentages of Gr-1^{high} (open bar), Gr-1^{low} (filled bar) cells. Data are shown as mean±SD. Student's t-test, ***P<0.001.

FIGS. 17A-17G. Increased myelopoiesis facilitated LPS-induced proteinuria. (17A-C) PBLs and BMCs were isolated from neutropenic (anti-Ly6G antibody-injected) and control (isotype antibody-injected) mice. (17A) Representative flow cytometry plots of PBLs and BMCs from control IgG or anti-Ly6G antibody injected mice. Myeloid cell populations (red circle) were gated based on their forward scatter (FSC) and side scatter (SSC) properties (n=4). (17B) The bar graph shows quantitative analysis of myeloid cell numbers measured by flow cytometry (n=4). (17C) ACR levels (n=5-7 per group from two independent experiments). Data are shown as mean±SEM. Student's t-test. **P<0.01, ***P<0.001. (17D-17G) BALB/c mice were treated with G-CSF (or PBS) for two consecutive days prior to LPS (or PBS) stimulation. Blood and bones (femurs and tibias) were collected 24 hours after LPS administration. (17D) PBLs and BMCs were isolated and labeled with fluorescence-conjugated antibodies specific for Gr-1. The bar graphs show percentages of Gr-1^{high} and Gr-1^{low} cells in the gated myeloid cell population (using forward and side scatter). (17E) The bar graph shows quantitative analysis of white blood cell (WBC) counts determined using a Hemavet (n=3). (17F) ACR levels. (17G) plasma suPAR levels. Data are shown as mean±SD (E) or ±SEM (17F and 17G). Student's t-test. *P<0.05, **P<0.01.

FIGS. 18A-18D. Proteinuria in Adriamycin (ADR) induced nephropathy was not associated with elevated suPAR levels as well as increased percentage of Gr-1^{lo} BM cells. (18A-18D) Male BALB/c mice were injected with ADR via the tail vein at a dose of 11 mg per kg body weight. Six days after ADR injection, urine and blood samples were collected and femurs, and tibias were harvested from the sacrificed mice. (18A) proteinuria. (18B) plasma suPAR levels. (18C) urinary suPAR levels. (18D) percentage of Gr-1^{lo} BM myeloid cells. Data are shown as mean±SEM. Student t-test, ***P<0.001, NS, not significant.

FIGS. 19A-19F. Kidney functions in the xenograft mice. (19A) Comparison of humanization methods between the original publication and current study. (19B) Human PBMCs were isolated from 2 different patients with recurrent FSGS and healthy donors, then injected into NSG mice. Engraftment rates of human cells were determined by the percentage of human CD45⁺ cells in blood and BM of the NSG mice at 12 weeks post engraftment (n=2 per group). (19C-19F) Non-depleted (whole) or CD34⁺ cell depleted (CD34Δ) PBMCs from patients with recurrent FSGS and normal individuals were transferred into NSG mice. Blood and kidney were harvested from the NSG mice on 10 weeks post-engraftment (n=2-5 per group). (19C) Sections of formalin-fixed kidney glomeruli from the xenograft mice were stained with H&E and with PAS. Scale bars, 50 μm. (19D) Blood urea nitrogen (BUN), as a marker for kidney function, was measured in serum samples from the humanized mice using a colorimetric-based assay kit (BioAssay Systems). (19E) Kidney weights. (19F) Representative SEM images of whole glomeruli of the xenograft mice. Scale bar, 10 μm.

FIGS. 20A-20C. GVHD is not a major cause of renal dysfunction observed in the xenograft mice. (20A) Body weights (g) of the xenograft mice. (20B) Representative immunofluorescent images of glomeruli stained with human IgG (green), synaptopodin (glomerular marker, red), and DAPI (blue). (20C) Representative images of H&E-stained skin and liver sections from the xenograft mice.

### DETAILED DESCRIPTION

The embodiments disclosed below are not intended to be exhaustive or to limit the scope of the disclosure to the precise form in the following description. Rather, the embodiments are chosen and described as examples so that others skilled in the art may utilize its teachings.

Embodiments of the present invention relate to methods of treating kidney diseases or disorders and methods of identifying agents for treatment of kidney diseases.

Here, “a subject in need of treatment” refers to a subject, including a human or other mammal, who is affected with a disorder characterized by proteinuria, is at risk for or is undergoing kidney failure, has received a kidney graft, or any combination thereof. A disorder characterized by proteinuria includes, for example, kidney or glomerular diseases, membranous glomerulonephritis, focal segmental glomerulonephritis, minimal change disease, nephrotic syndromes, pre-eclampsia, eclampsia, kidney lesions, collagen vascular diseases, stress, strenuous exercise, benign orthostatic (postural) proteinuria, focal segmental glomerulosclerosis, IgA nephropathy, IgM nephropathy, membranoproliferative glomerulonephritis, membranous nephropathy, end-stage kidney disease, sarcoidosis, Alport's syndrome, diabetes mellitus, kidney damage due to drugs, Fabry's disease, infections, aminoaciduria, Fanconi syndrome, hypertensive nephrosclerosis, interstitial nephritis, sickle cell disease, hemoglobinuria, multiple myeloma, myoglobinuria, cancer, Wegener's granulomatosis, and glycogen storage disease type 1.

As used herein, “proteinuria” refers to proteins passing through podocytes that have suffered damage or through a podocyte-mediated barrier that normally would not allow protein passage. Such structural damage may be visualized in vitro or in vivo. In the body of a subject, “proteinuria” may refer to the presence of an excessive amount of serum protein (e.g., albumin) in urine. Proteinuria may be a symptom of renal, urinary, and nephrotic syndromes (i.e., proteinuria larger than 3.5 grams per day), eclampsia, toxic lesions of kidneys, pancreatic distress, and it is frequently a symptom of diabetes mellitus. With severe proteinuria, general hypoproteinemia can develop and it results in diminished oncotic pressure (ascites, edema, hydrothorax).

Proteinuria can be primarily caused by one or more alterations of structural proteins involved in the cellular mechanism of filtration. The pathophysiological causes of proteinuria can be divided in the following major groups: (1) genetically determined disturbances of the structures which form the “glomerular filtration unit” like the glomerular basement membrane, the podocytes, or the slit diaphragm; (2) inflammatory processes, either caused directly by autoimmune processes or induced indirectly by microbes; (3) damage of the glomeruli caused by agents; or (4) as the final result of progressive tubulointerstitial injury finally resulting in the loss of function of the entire nephron.

“Myeloid progenitor cell” refers to a multipotent or unipotent progenitor cell capable of ultimately developing into any of the terminally differentiated cells of the myeloid lineage, but which do not typically differentiate into cells of the lymphoid lineage. Hence, “myeloid progenitor cell” refers to any progenitor cell in the myeloid lineage. Committed progenitor cells of the myeloid lineage include oligopotent CMP, GMP, and MEP as defined herein, but also encompass unipotent erythroid progenitor, megakaryocyte progenitor, granulocyte progenitor, and macrophage progenitor cells. Different cell populations of myeloid progenitor cells are distinguishable from other cells by their differentiation potential, and the presence of a characteristic set of cell markers. In some embodiments, the myeloid progenitor cells are bone marrow myeloid progenitor cells.

“Treating”, “treat”, or “treatment” within the context of the instant invention, means an alleviation of symptoms associated with a disorder or disease, or halt of further progression or worsening of those symptoms, or prevention or prophylaxis of the disease or disorder. In some embodiments, successful treatment may include an alleviation of symptoms related to a kidney disease or disorder. For example, within the context of this invention, successful treatment may include an alleviation of symptoms related to a kidney disease or disorder or a halting in the progression of a kidney disease or disorder. Likewise, a therapeutically effective amount of a compound is a quantity sufficient to diminish or alleviate at least one symptom associated with the conditions being treated. “Therapeutically effective amount” refers to the quantity of a component which is sufficient to yield a desired therapeutic response without undue adverse side effects (such as toxicity, irritation, or allergic response) commensurate with a reasonable benefit/risk ratio when used in the manner of this invention.

As used herein, the term “test substance” or “candidate therapeutic agent” or “agent” are used interchangeably herein, and the terms are meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, antibodies, enzymes, peptides organic or inorganic molecules, natural or synthetic compounds and the like. A test substance or agent can be assayed in accordance with the methods of the invention at any stage during clinical trials, during pre-trial testing, or following FDA-approval.

Methods of Treatment

Methods of treatment of a kidney disease or disorder are provided. The methods include administering to a subject in need thereof an effective amount of an agent which inhibits myeloid progenitor cells in the subject from producing soluble urokinase receptor (suPAR).

uPAR is a glycosylphosphatidylinositol (GPI)-anchored protein with three extracellular domains. Cleavage of the GPI anchor generates suPAR. suPAR has been found elevated in sera of patients with kidney disease.

In some embodiments, the agent removes CD34+ cells from the subject. As used herein, the term “CD34” refers to a cell surface marker found on certain hematopoietic and non-hematopoietic stem cells, and having the gene symbol CD34. The terms “depleting” and “removing” refer to the removal of a majority (i.e., more than one-half) of a particular type of cell (e.g., CD34+) from a sample.

The removal of CD34+ cells in accordance with the present invention is in some embodiments accomplished using an immunologic technique and, in some embodiments, involves treating the subject with CD34 antibodies. In some embodiments, the CD34 antibodies are attached to magnetic beads, which enable separation of CD34+ cells from the subject, for example from the blood using a magnetic cell separator. By way of non-limiting example, magnetic beads for use in accordance with the present invention include the super-paramagnetic micro-beads sold by Miltenyi Biotec Inc., Auburn, Calif. Other methods of removing CD34+ cells may also be used.

In some embodiments, the method of treatment includes identifying subjects for administration of the agent. Identifying the subjects may include isolating myeloid progenitor cells from the subject. In some embodiments, the myeloid progenitor cells may be isolated from the subject by collecting a blood sample or other tissue sample from the subject. In a blood sample, peripheral blood mononuclear cells may be isolated using any technique known to one skilled in the art. In some embodiments, the myeloid progenitor cells may be enriched for CD34+ cells using techniques similar to those described above with antibodies and magnetic beads and retaining the CD34+ cells.

In some embodiments, the isolated myeloid progenitor cells may be transferred to a murine host. In some embodiments, the murine host may be an immunocompromised host. The number of Gr-1^{lo} cells may be measured in the murine host after the cells have been transferred to the host. In some embodiments, the number of Sca-1⁺/Gr-1^{lo} cells may be measured in the murine host after the cells have been transferred to the host. In some embodiments, the measurement may be hours, days, weeks or months after the transfer of the cells to the host. The number of Gr-1^{lo} cells are compared to a control number of cells. The therapeutic agent is administered to subjects whose myeloid progenitor cells give rise to an increased number of Gr-1^{lo} cells in the murine host relative to the murine control.

In some embodiments, the agent may be an antibody, aptamer, antisense oligonucleotide, a natural agent, a synthetic agent or combinations thereof. In some embodiments, the agent is a chemical compound, natural or synthetic, in particular an organic or inorganic molecule of plant, bacterial, viral, animal, eukaryotic, synthetic or semisynthetic origin, capable of inhibiting myeloid progenitor cells from producing soluble urokinase receptor.

In some embodiments, the treatment may include an oral administration of a compound. In some embodiments, the dose of the compound administered to the subject may be in the range from about 500 mg to 2000 mg per day for patients. In some embodiments, the dose of the compound to be administered alone or in combination therapy warm-blooded animals, for example humans, is preferably from approximately 0.01 mg/kg to approximately 1000 mg/kg, more preferably from approximately 1 mg/kg to approximately 100 mg/kg, per day, divided preferably into 1 to 3 single doses which may, for example, be of the same size. Usually children receive half of the adult dose, and thus the preferential dose range for the inhibitor in children is 0.5 mg/kg to approximately 500 mg/kg, per day, divided preferably into 1 to 3 single doses that may be of the same size.

A compound can be administered alone or in combination with another autophagy activators, possible combination therapy taking the form of fixed combinations or the administration of a compound and another inhibitor being staggered or given independently of one another. Long-term therapy is equally possible as is adjuvant therapy in the context of other treatment strategies, as described above. Other possible treatments are therapy to maintain the subject's status after symptom amelioration, or even preventive therapy, for example in subjects at risk.

Effective amounts of the compounds described herein generally include any amount sufficient to detectably ameliorate one or more symptoms of a neurodegenerative disorder, or by detecting an inhibition or alleviation of symptoms of a kidney disease or disorder. The amount of active ingredient that may be combined with the carrier materials to produce a single dosage form will vary depending upon the host treated and the particular mode of administration. It will be understood, however, that the specific dose level for any particular subject will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

According to the methods of treatment of the present invention, a kidney disease or disorder is reduced or prevented in a subject such as a human or lower mammal by administering to the subject an amount of an agent, in such amounts and for such time as is necessary to achieve the desired result.

It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts.

Compositions for administration of the active agent in the method of the invention may be prepared by means known in the art for the preparation of compositions (such as in the art of veterinary and pharmaceutical compositions) including blending, grinding, homogenising, suspending, dissolving, emulsifying, dispersing and where appropriate, mixing of the active agent, together with selected excipients, diluents, carriers and adjuvants.

For oral administration, the composition may be in the form of tablets, lozenges, pills, troches, capsules, elixirs, powders, including lyophilised powders, solutions, granules, suspensions, emulsions, syrups and tinctures. Slow-release, or delayed-release, forms may also be prepared, for example in the form of coated particles, multi-layer tablets or microgranules.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methyl cellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further include dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further include one or more emulsifying agents. Suitable emulsifying agents include dispersing agents as exemplified above or natural gums such as guar gum, gum acacia or gum tragacanth.

Methods of Identifying an Agent

Methods of identifying an agent for treatment of kidney diseases or disorders is disclosed. The method includes administering the agent to a murine host. The murine host has an increased number of Gr-1^{lo} cells or in some embodiments an increased number of Sca-1⁺/Gr-1^{lo}. The effect of the agent on an indicator of kidney disease is determined and the agent is identified as useful for treatment when the agent ameliorates the indicator of kidney disease in the host relative to a control. In some embodiments, the indicator of the kidney disease or disorder is an albumin-to-creatinine ratio (ACR), an amount of suPAR, a proportion of Gr-1^{high} to Gr-1^{lo} cells, or a podocyte foot process effacement measurement, although other indicators of kidney diseases or disorders may also be used. In some embodiments the method may include administering myeloid progenitor cells from a subject having FSGS to the murine host to increase the number of Gr-1^{lo} cells relative to a control. In some embodiments, the myeloid progenitor cells may be enriched for CD34+ cells.

The test compounds of the present invention can be obtained using any of the numerous approaches known in the art. In some embodiments, the test compound is a member of a library of test compounds. A “library of test compounds” refers to a panel comprising a multiplicity of test compounds. An approach for the synthesis of molecular libraries of small organic molecules has been described (Angew. Chem. Int. Ed. Engl. 33:2059; Carell et al. (1994) Angew. Chem. Int. Ed. Engl. 33:2061). The compounds of the present invention can be obtained using any of the numerous approaches in combinatorial library methods known in the art, including: biological libraries; spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). Other exemplary methods for the synthesis of molecular libraries can be found in the art, for example in: Erb et al. (1994). Proc. Natl. Acad. Sci. USA 91:11422; Horwell et al. (1996) Immunopharmacology 33:68-; and in Gallop et al. (1994); J. Med. Chem. 37:1233-. Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310). In still another embodiment, the combinatorial polypeptides are produced from a cDNA library. Exemplary compounds that can be screened for activity include, but are not limited to, peptides, nucleic acids, carbohydrates, small organic molecules, and natural product extract libraries.

Discussion

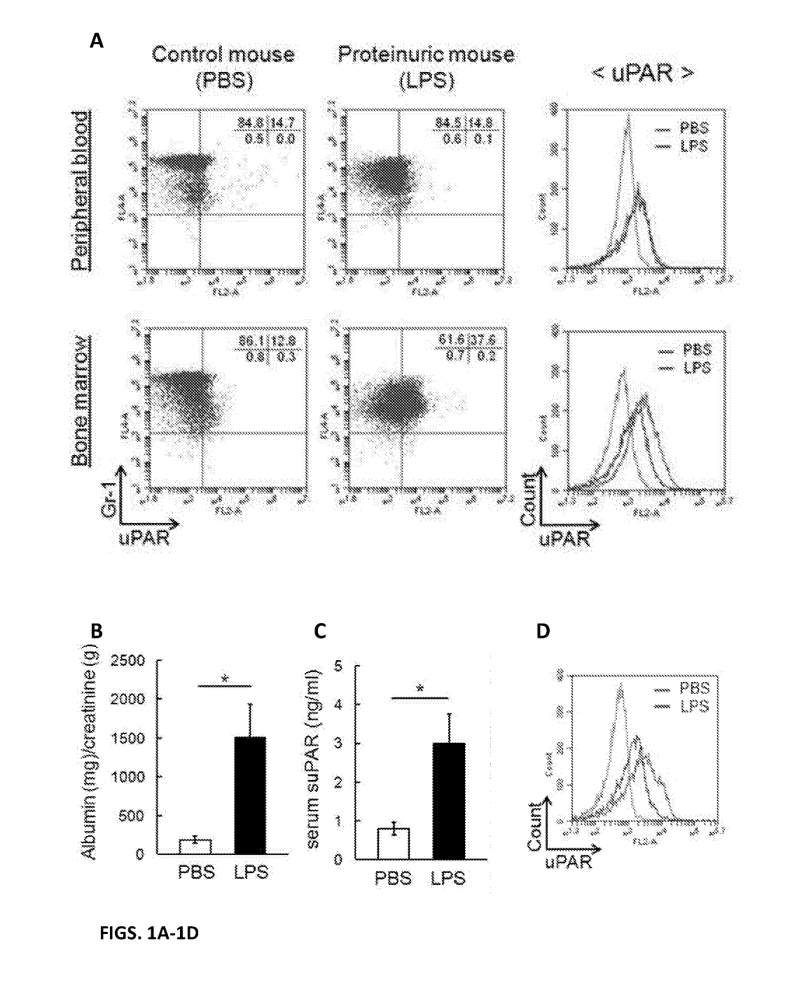

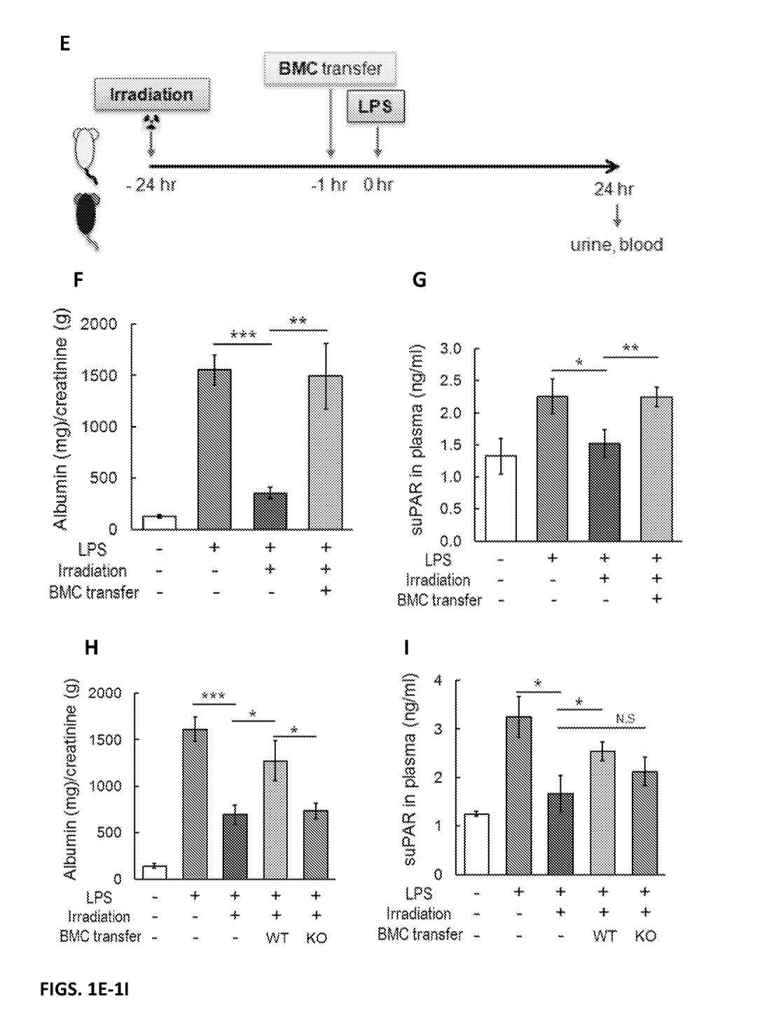

To identify the origin of pathogenic suPAR in our lipopolysaccharide (LPS) proteinuric mouse model of FSGS, uPAR expression was studied in hematopoietic cells, including peripheral blood leukocytes (PBLs) and bone marrow cells (BMCs), since increased expression and secretion of (s)uPAR has previously been observed in activated neutrophils (Pliyev et al, 2009; Pliyev, Menshikow.), monocytes (Dekkers et al, 2000), and hematopoietic stem/progenitor cells (HSPCs) (Tjwa et al, 2009). Compared to phosphate buffered saline (PBS)-treated control mice, only myeloid (Gr-1⁺) cells from bone marrow (BM), but not from peripheral blood (PB), increased uPAR expression in the LPS-induced proteinuric mice (FIG. 1A). To evaluate if BM myeloid cells may participate in suPAR-mediated proteinuria, we injected PBS or LPS into NOD-scid IL2rγ^{null} (NSG) mice that lack mature T, B, and NK cells, but have unimpaired production of myeloid cells (Shultz et al., 2005). Indeed, LPS injection caused proteinuria (FIG. 1B), enhanced blood suPAR levels (FIG. 1C), and increased uPAR expression on BM myeloid cells (FIG. 1D) in these mice. These data are consistent with BM myeloid cells as a source of pathogenic suPAR.

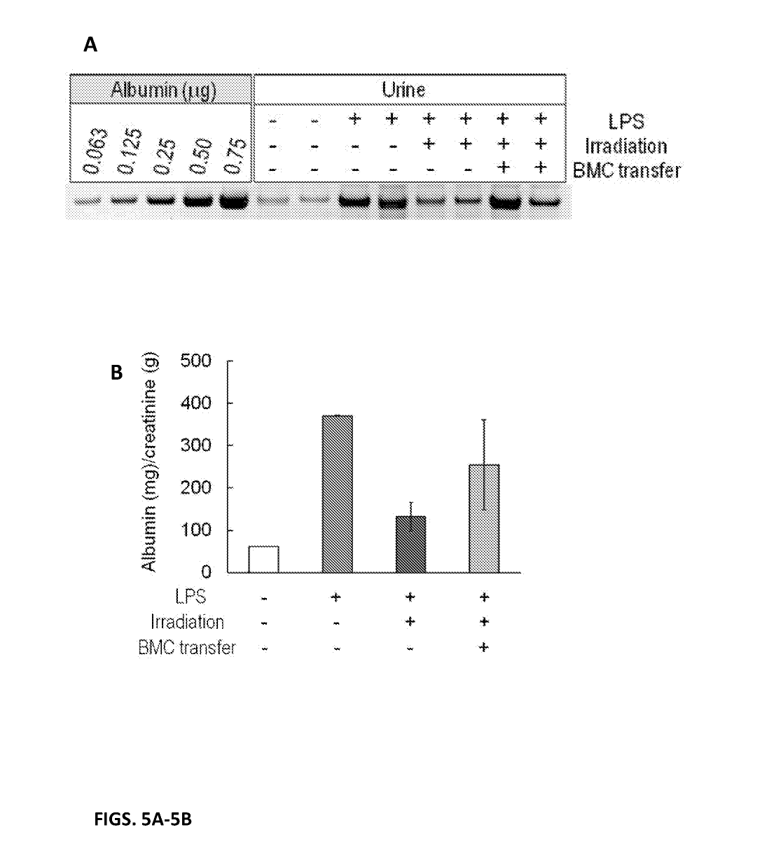

To determine if the myeloablation prevents proteinuria, BALB/c mice were irradiated prior to LPS injection (FIG. 1E). The irradiated mice showed a significant decrease in the degree of proteinuria (FIG. 1F, and FIGS. 5A and 5B) and in plasma suPAR levels (FIG. 1G). However, transfer of normal BMCs into recipient mice following irradiation, but prior to LPS-injection, again resulted in proteinuria with elevated plasma suPAR levels (FIGS. 1F and 1G). Therefore, BMCs are necessary for LPS induced proteinuria and associated increases in plasma suPAR.

To better understand the role of BMC-derived suPAR in the development of proteinuria, the BMC transfer experiment was repeated comparing the bone marrow from wild-type C57BL/6 mice with syngeneic uPAR deficient (Plaur^{-/-}) mice. As shown above, irradiation significantly reduced proteinuria and limited production of suPAR in response to LPS, whereas transfer of WT but not Plaur^{-/-} BMCs could restore proteinuria in irradiated recipients (FIGS. 1H and 1I). Plasma suPAR levels were also partially restored by the transfer of WT BMCs, while transfer of Plaur^{-/-} BMCs showed a mild, but non-significant increase in suPAR levels compared to irradiated mice that did not receive any BMCs (FIG. 1I). Due to strain-specific differences in radiation sensitivity between B6 (resistant) and BALB/c (sensitive) mice (Duran-Struuck et al., 2009), we postulate that residual WT BMCs in the irradiated B6 mice are the source of the small increase in plasma suPAR seen in response to LPS in this model.

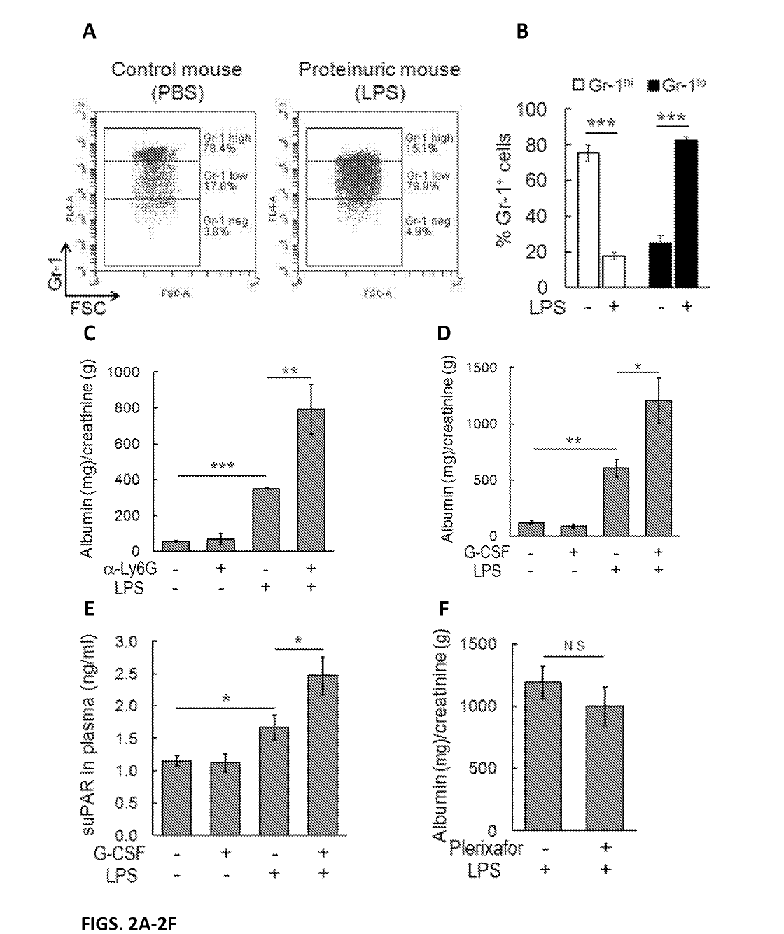

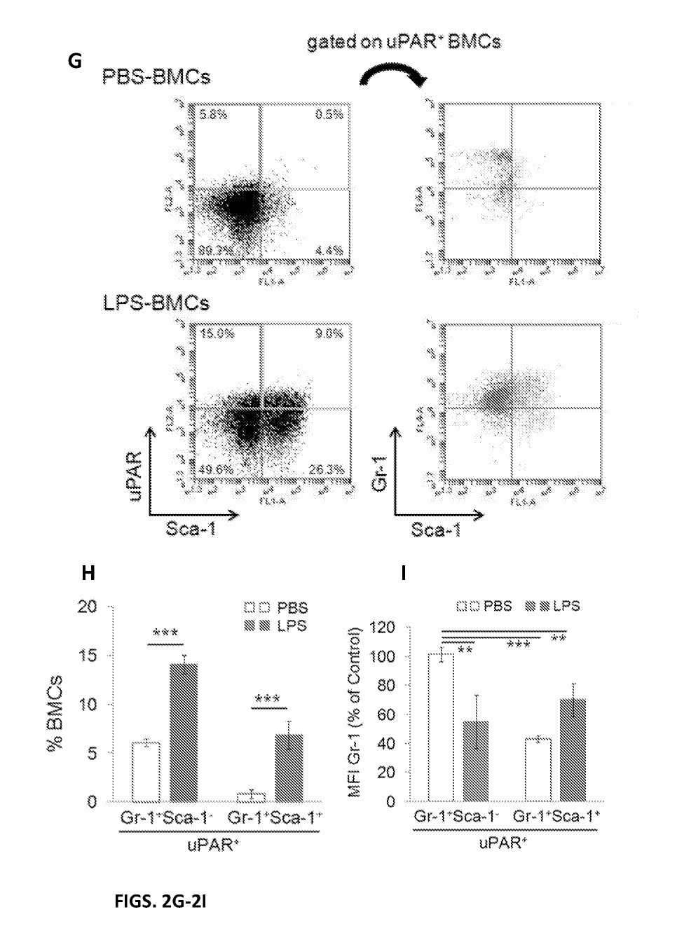

In addition to increased uPAR expression on BM myeloid (mostly Gr-1^{lo}) cells, we also observed markedly increased ratio of Gr-1^{lo} (less mature or immature) to Gr-1^{hi} (mature) BM myeloid cells in this LPS-induced proteinuric mice, compared to PBS-treated control mice (FIGS. 2A and 2B). We therefore hypothesized that the reactive expansion of immature myeloid cell populations in BM may contribute to the pathogenic kidney process. We tested this with eradication of peripheral mature neutrophils in vivo using anti-Ly6G antibody and observed increased proteinuria in LPS-challenged mice (FIG. 2C, and FIGS. 5A and 5B). Similarly, treatment with G-CSF to stimulate myelopoiesis (FIGS. 5C-5E), accelerated proteinuria, and enhanced plasma suPAR levels, compared to LPS alone (FIGS. 2D and 2E). Because G-CSF enhances myelopoiesis, but can also promote HSPC mobilization from the BM into the circulation (Basu et al., 2002), we examined whether HSPC mobilization alone could facilitate proteinuria in LPS-challenged mice. To address this possibility, we injected plerixafor, a CXCR4 antagonist and HSPC mobilizer, prior to challenge with LPS. Unlike G-CSF, treatment with plerixafor did not enhance proteinuria (FIG. 2F). Therefore, uPAR-expressing BM immature myeloid cells constitute the major source of suPAR and are necessary for the development of proteinuria.

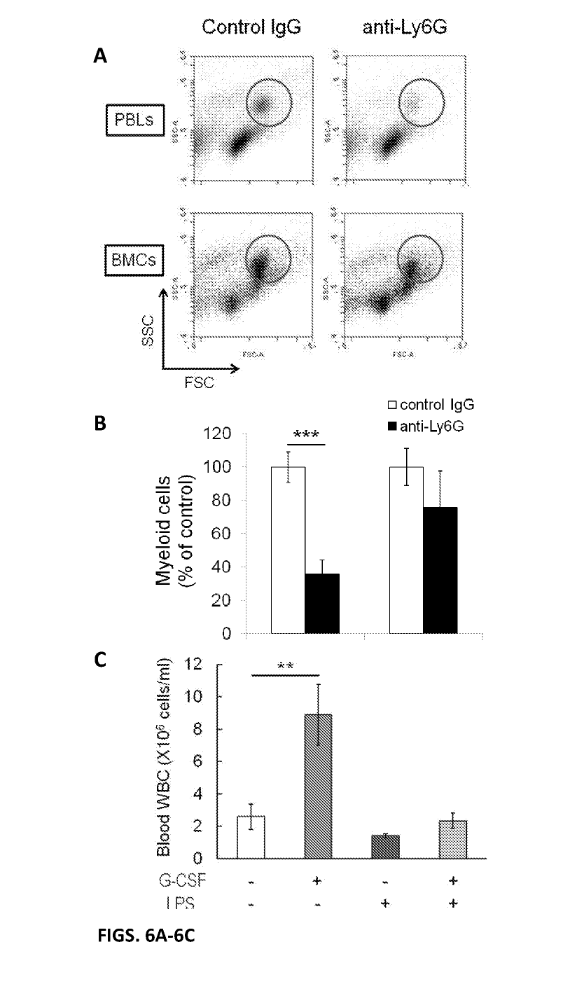

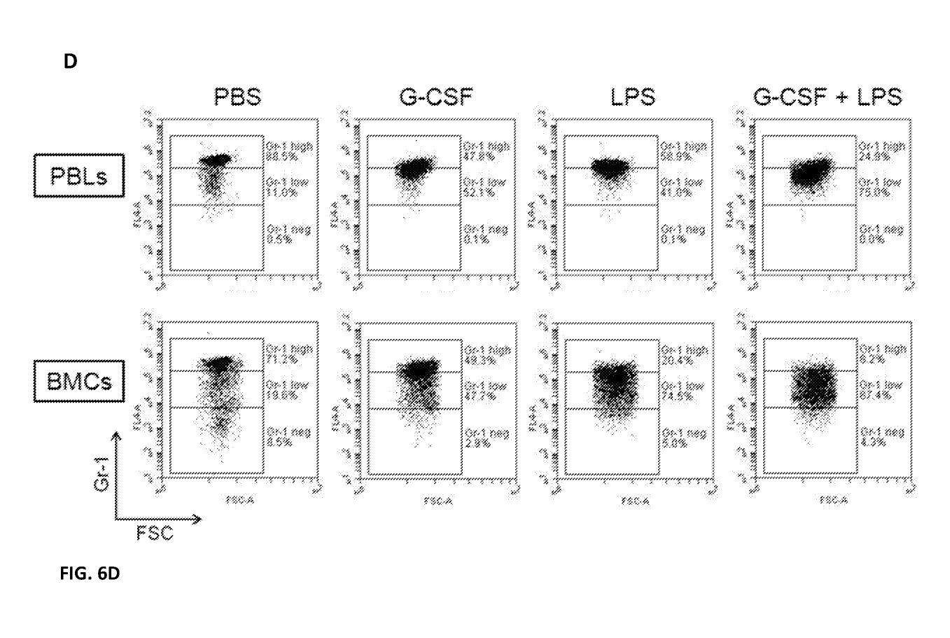

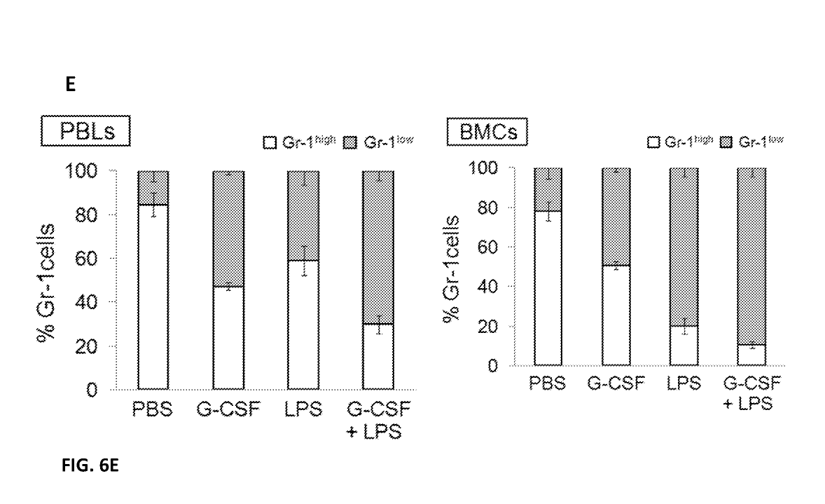

Consistent with a previous report (Tjwa et al., 2009), Plaur^{-/-} mice exhibited a blunted expansion of the BM Sca-1⁺ population in response to LPS (FIG. 6A), as compared to WT mice. Stem cell antigen-1 (Sca-1) is expressed on HSPC populations among other cell types and is highly responsive to proliferative stimuli. To investigate the role of the BM Sca-1⁺ population in suPAR-mediated proteinuria, we first examined Sca-1 expression in uPAR-expressing BMCs. We observed that treatment with LPS increased the population of Sca-1⁺Gr-1^{lo} myeloid progenitor cells (MPCs) among uPAR-expressing BMCs (FIGS. 2G-2I).

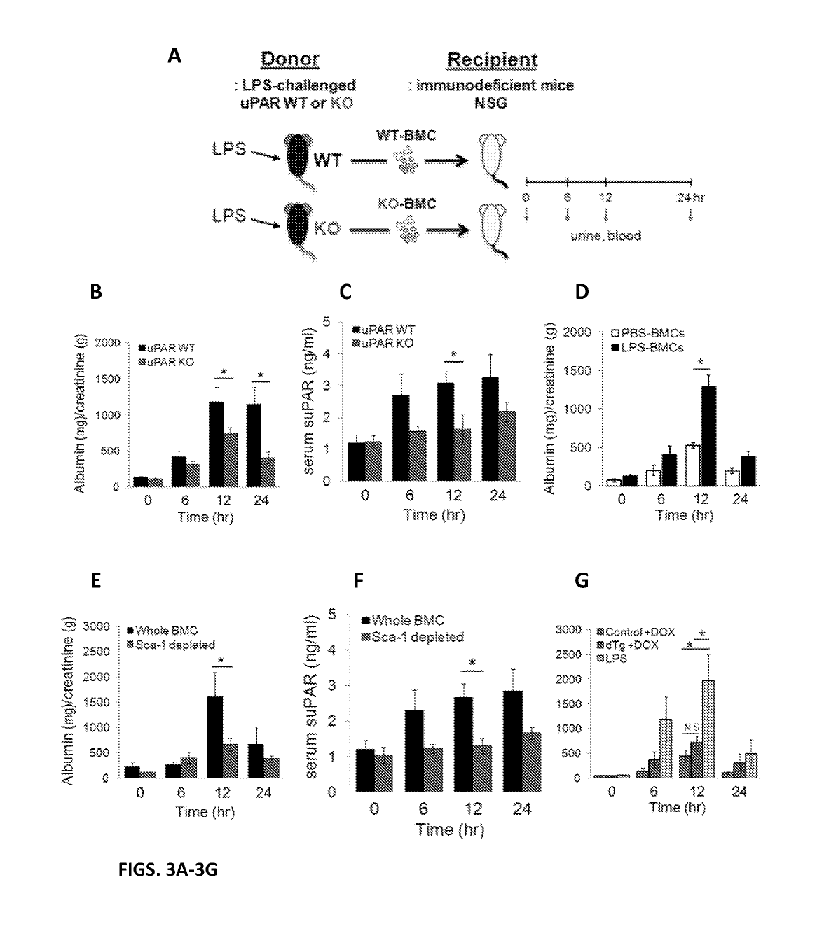

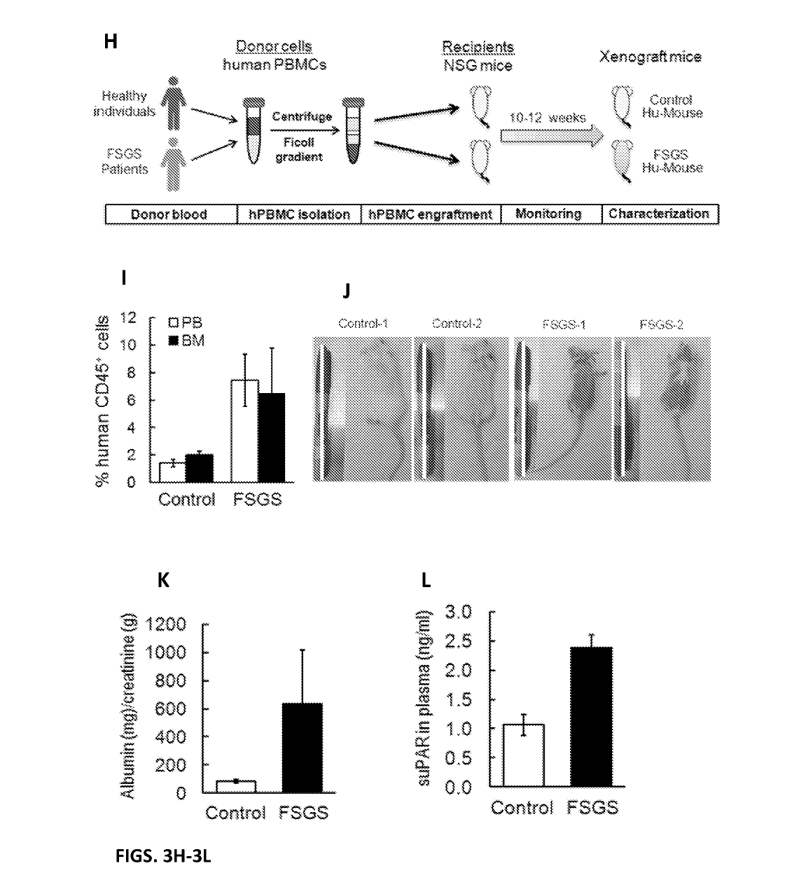

Next, we hypothesized that if suPAR is the cause of the disease, not simply a consequence, then cellular sources of the pathogenic suPAR could propagate the disease in healthy mice when they are transferred. To address this, we assessed the adoptive transfer of BMCs of LPS-challenged donor mice into immunocompromised NSG recipient mice (FIG. 3A). Transfer of LPS-challenged WT BMCs induced proteinuria and suPAR levels (FIGS. 3B and 3C). Of note, these effects were abrogated when Plaur^{-/-} BMCs were transferred (FIGS. 3B and 3C). Similarly, the recipient NSG mice developed proteinuria when donor NSG mice were challenged with LPS, but not PBS, prior to transfer suggesting that BM myeloid cells are sufficient for the disease propagation (FIG. 3D).

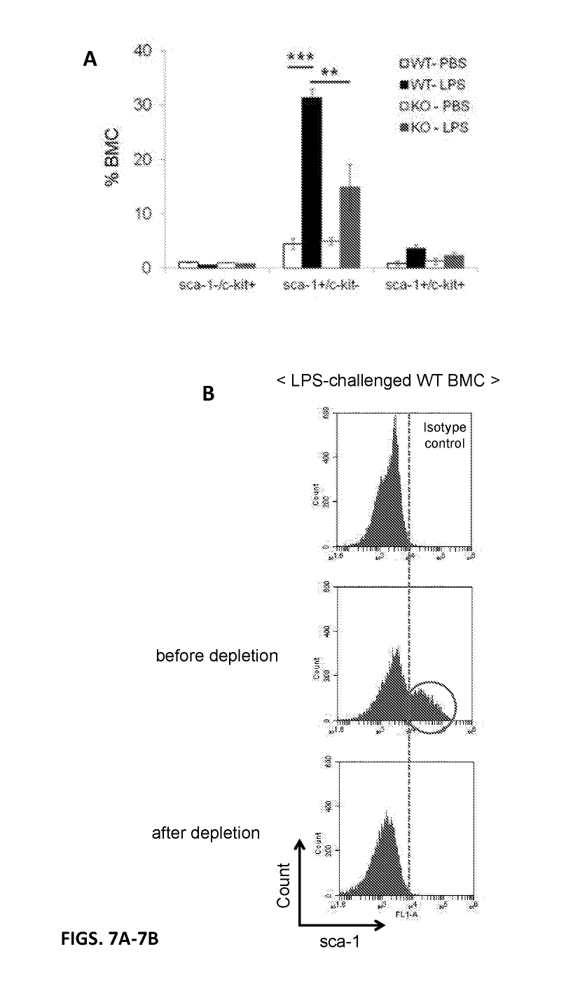

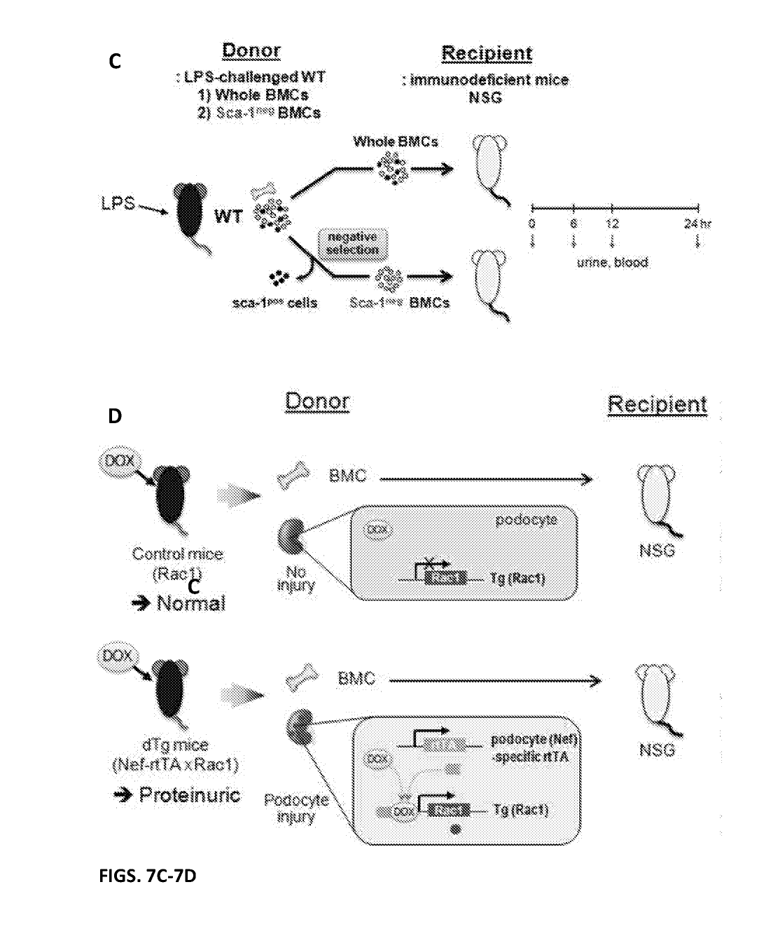

To further evaluate if the Sca-1⁺Gr-1^{lo} MPCs can transfer disease when injected into healthy mice, Sca-1⁺ cells were depleted from LPS-challenged BMCs by magnetic separation prior to transfer into NSG mice (FIGS. 7B and 7C). The removal of Sca-1⁺ BMCs lowered both proteinuria (FIG. 3E) and suPAR levels (FIG. 3F), compared to non-depleted control BMCs. To assess whether proteinuria triggered by alternative means secondarily alters hematopoietic cells and thus makes them proteinuria-inducing, we adoptively transferred BMCs from mice with proteinuria triggered by kidney podocyte injury (FIG. 7D). Transgenic mice (Nef-rtTAxRac1) that express constitutively active Rac1 specifically in podocytes following induction with doxycycline (DOX), demonstrate heavy proteinuria (Yu et al.). BMCs from these proteinuric mice were transferred into NSG recipient mice and did not cause proteinuria. In contrast, BMCs from LPS-proteinuric mice induced proteinuria (FIG. 3G). Taken together, these results emphasize the important, primary role of BM MPCs in regulation of suPAR production and proteinuria development.

Given that the CD34⁺ cells of patients with FSGS or the murine BMCs/HSPCs from mice with FSGS can transfer the disease (and resultant proteinuria) upon introduction into healthy mice (Sellier-Leclerc et al., 2007; Nishimura et al, 1994), we tested whether PBMCs of patients with FSGS can induce proteinuria in normal mice. PBMCs from patients with FSGS and from normal individuals were injected into NSG mice. These immunodeficient mice were monitored for the development of disease to determine if they showed similar manifestations to those observed in patients with FSGS (FIG. 3H). We found human (hCD45⁺) cells remaining in blood and BM of the NSG mice at 12 weeks post engraftment (FIG. 3I). Indeed, engraftment of PBMCs from two different FSGS patients into NSG mice resulted in proteinuria (FIG. 3K) and elevated mouse suPAR levels in the blood (FIG. 3L), whereas none of the mice engrafted with healthy PBMCs showed proteinuria 12 weeks after engraftment.

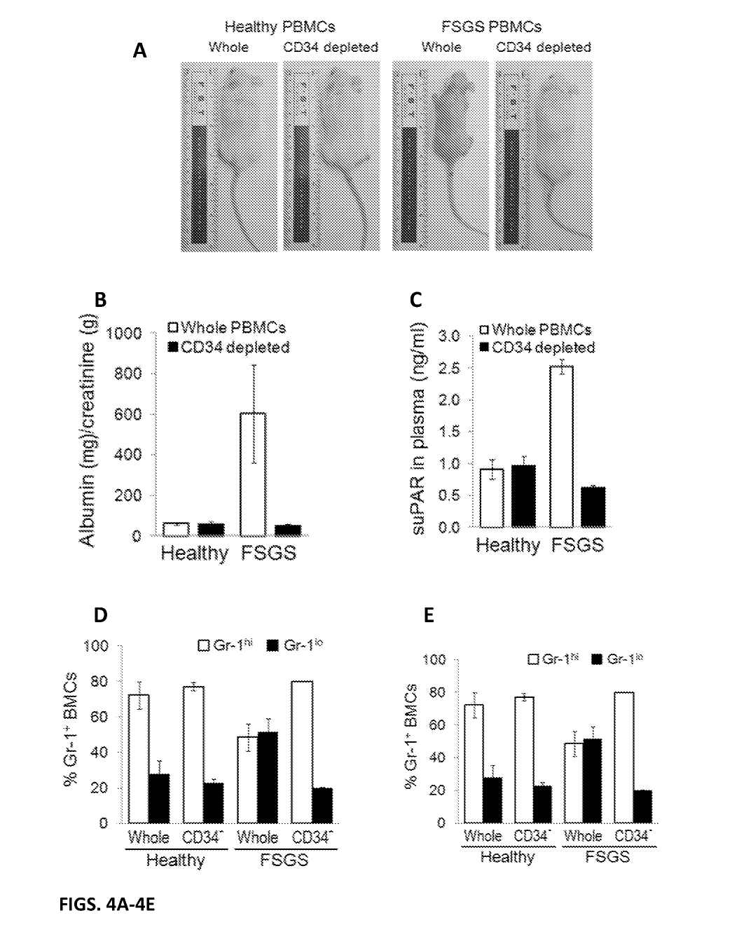

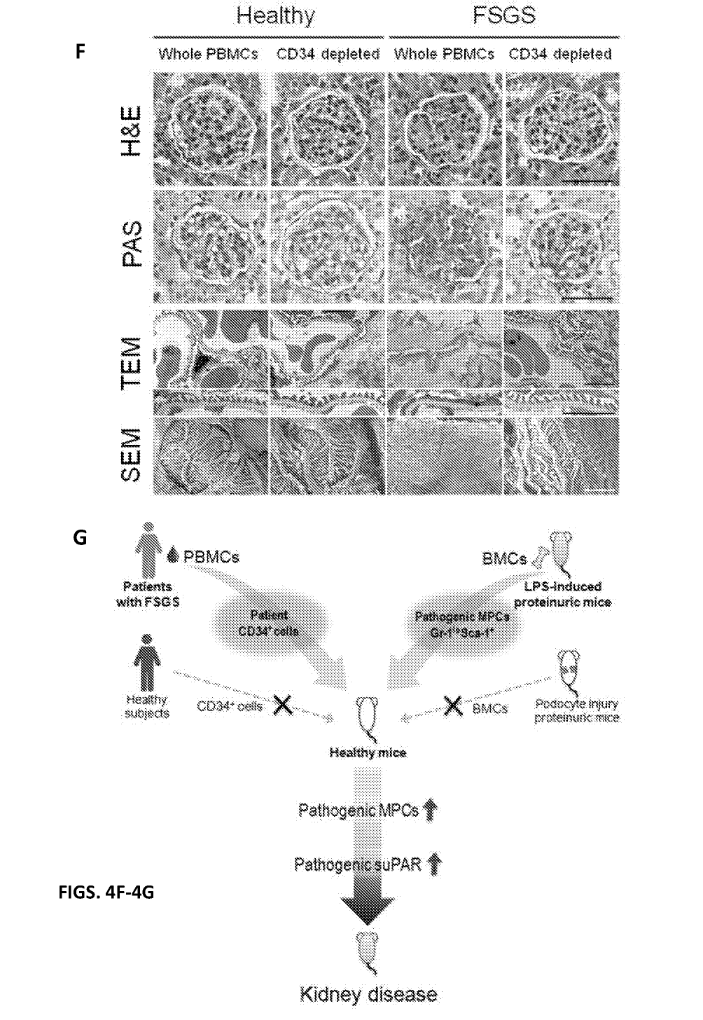

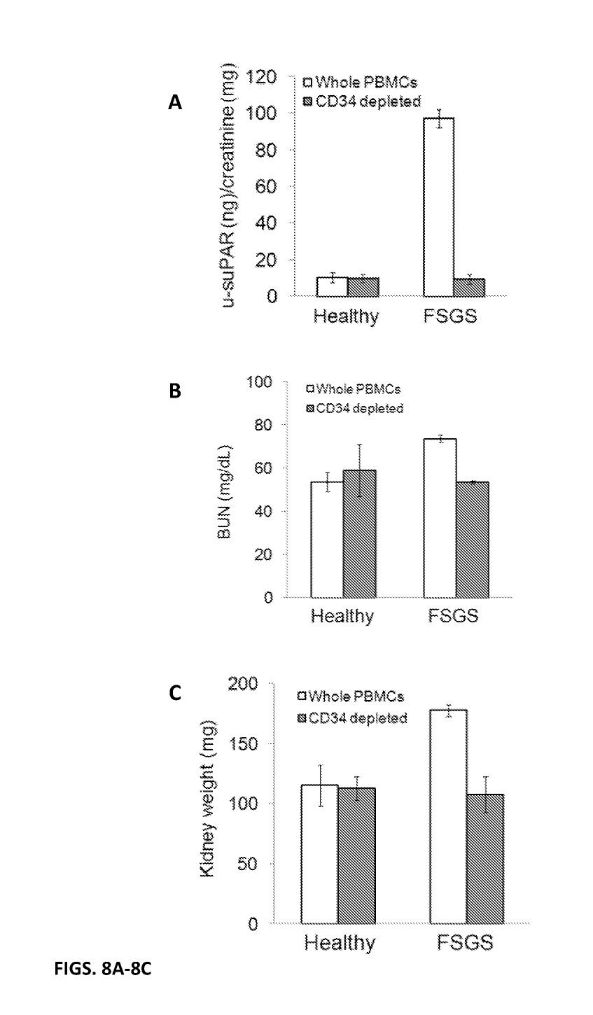

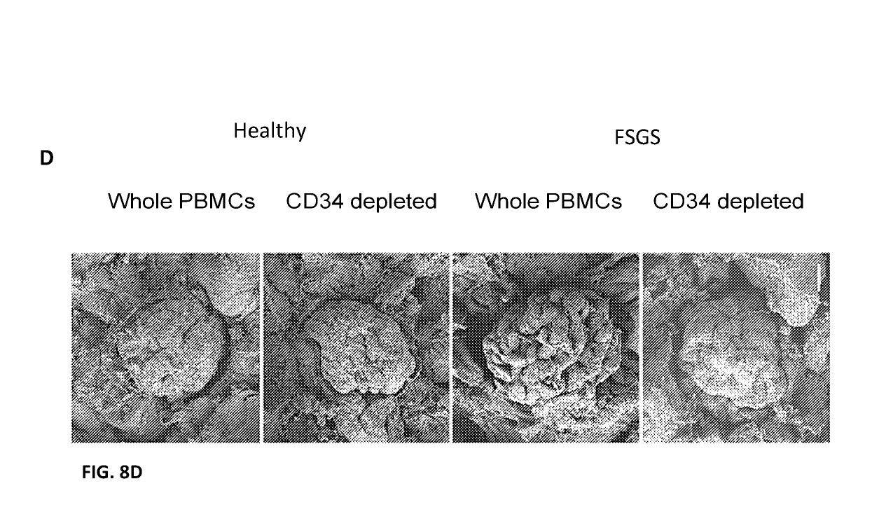

Using this xenograft disease model, we further investigated the role of mouse MPCs in production of pathological suPAR and development of proteinuria. Although Sca-1 is a murine-specific cell surface marker without a known human homologue, the CD34 antigen is widely used to identify the fraction of immature BMCs in humans. Whole PBMCs, or PBMCs depleted of CD34⁺ cells (CD34⁻) derived from patients with FSGS and normal individuals were transferred into NSG mice. Notably, the mice receiving patient CD34⁺ PBMCs, but not CD34⁻ PBMCs, developed proteinuria (FIG. 4B) in association with high plasma suPAR levels (FIG. 4C). The BM of these mice increased Gr-1^{lo} population (FIG. 4D) and also enhanced uPAR expression on the myeloid cells (FIG. 4E) as seen in the LPS-proteinuric mouse model. Histologically, mild glomerulosclerosis was observed in the mice that received FSGS PBMCs, although most glomeruli appeared normal (FIG. 4F). Electron microscopy studies revealed changes in glomerular structure of the mice that received FSGS PBMCs, with extensive podocyte foot process effacement (fusion) (FIG. 4F), implying disrupted kidney filter function. Consistent with these observations, these mice also exhibited enhanced blood urea nitrogen (BUN) levels, increased kidney weight, and enlarged, loosely structured glomeruli (FIGS. 8A-8C). Notably, the NSG mice receiving CD34⁻ PBMCs from FSGS patients had no evidence of FSGS, indicating that patient CD34⁺ cells are key regulators in the pathogenesis of FSGS. This is surprising given the rarity of CD34⁺ cells among PBMCs, present at a frequency of only 0.05˜0.2%. Consistent with previous observations ((Sellier-Leclerc et al., 2007; Nishimura et al, 1994), we demonstrated that the FSGS CD34⁺ cells are critical for the induction of FSGS. Our results suggest that patient CD34⁺ cells may trigger an expansion of mouse pathogenic Gr-1^{lo} MPCs, leading to increased production of suPAR (presumably pathological forms) that cause FSGS (FIG. 5G). The mechanism by which CD34⁺ cells from FSGS patients triggers an expansion of the pathogenic murine BMCs, resulting in proteinuria, remains unknown.

This study demonstrates that FSGS may originate as a primary BM progenitor cell disorder. Two analyzed model systems, murine LPS stimulation and human derived CD34⁺ PBMCs converge at the expansion of pathogenic MPCs. These Sca-1⁺Gr-1^{lo} cells result in a systemic release of kidney-pathogenic suPAR causing the podocytopathy that is characteristic of human FSGS. In addition to help explain the causes of FSGS, these findings also serve as a prototype for the pathogenesis of other diseases in which BM progenitors may regulate organ function via soluble mediators. In this regard, other ‘idiopathic’ disorders may evolve through similar or different mediators and diverge from the classical inflammatory response.

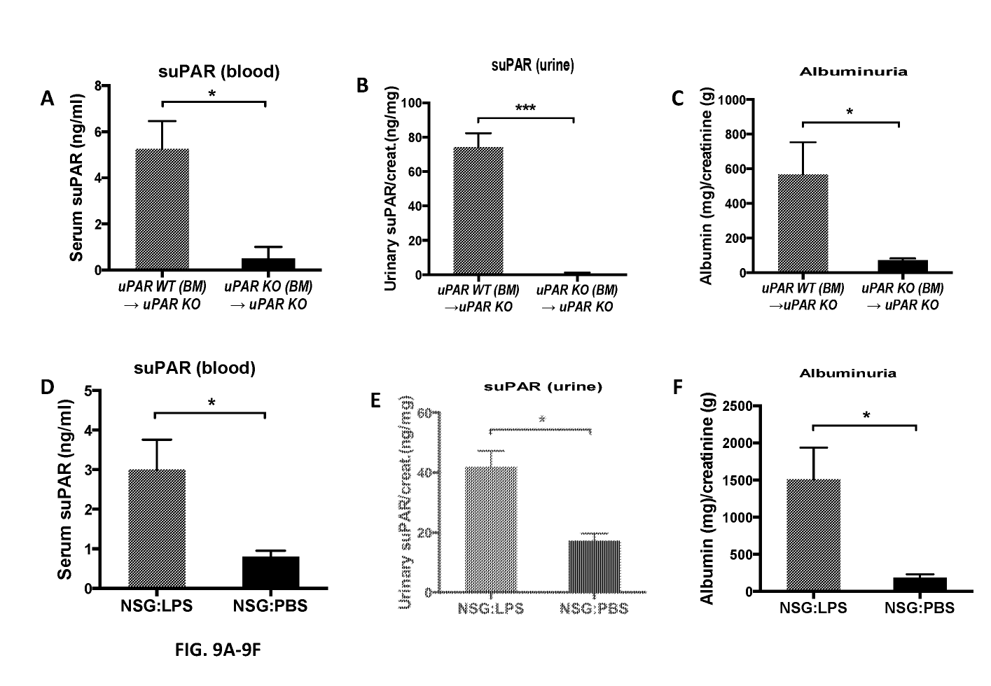

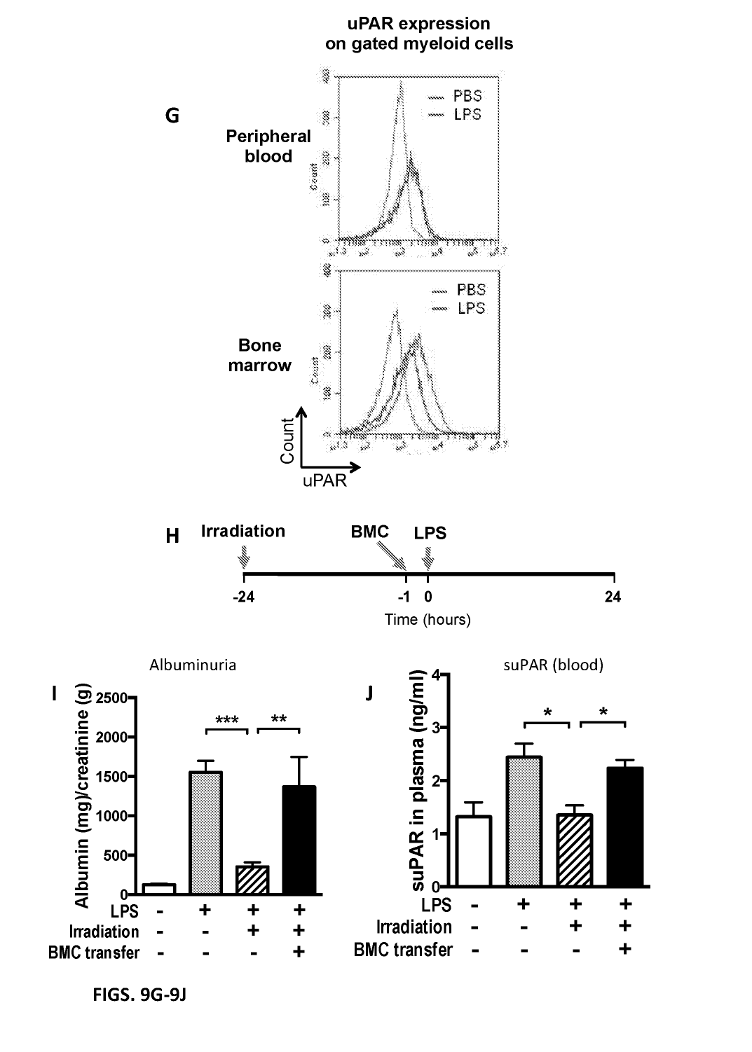

To evaluate the role of hematopoietic cells in suPAR production and proteinuria, bone marrow (BM) chimeric mice were generated. uPAR deficient (Plaur^{-/-}, KO) mice were irradiated and reconstituted with BM cells of either uPAR wild-type (Plaur^{+/+}, WT) or KO mice. All chimeric mice (WT→KO and KO→KO) showed successful engraftment rate of donor cells 6 weeks after transplantation (FIG. S2, WT→KO; 94.6%±3.7, KO→KO; 99.3%±0.7). As expected, KO→KO chimeric mice showed a strong defect in suPAR production (FIGS. 1, A and B) with lack of proteinuria development (FIG. 1C) upon LPS stimulation. In contrast, the chimeric mice expressing uPAR selectively within hematopoietic cells (WT→KO) exhibited elevated suPAR levels in both blood and urine (FIGS. 9, A and B), as well as proteinuria (FIG. 9C). These results suggest that hematopoietic cells are essential for the production of suPAR and the development of proteinuria in this model.

Given that hematopoietic cells are categorized into myeloid cells and lymphoid cells, we next examined which cell lineages participate in suPAR-mediated proteinuria, using NOD-scid IL2rγ^{null} (NSG) mice that lack mature lymphocytes, but have unimpaired production of myeloid cells (Shultz et al.). Despite impaired lymphoid populations, LPS stimulation elevated suPAR levels in both blood and urine (FIGS. 9, D and E), resulting in proteinuria (FIG. 9F). These data suggest that myeloid cells, but not lymphoid cells, are required for suPAR-mediated proteinuria. We next studied uPAR expression on myeloid cells in BM as well as in peripheral blood. In LPS treated animals, BM myeloid cells but not peripheral myeloid cells, exhibited elevated levels of uPAR on their membrane when compared to phosphate buffered saline (PBS)-treated mice (FIG. 9G).

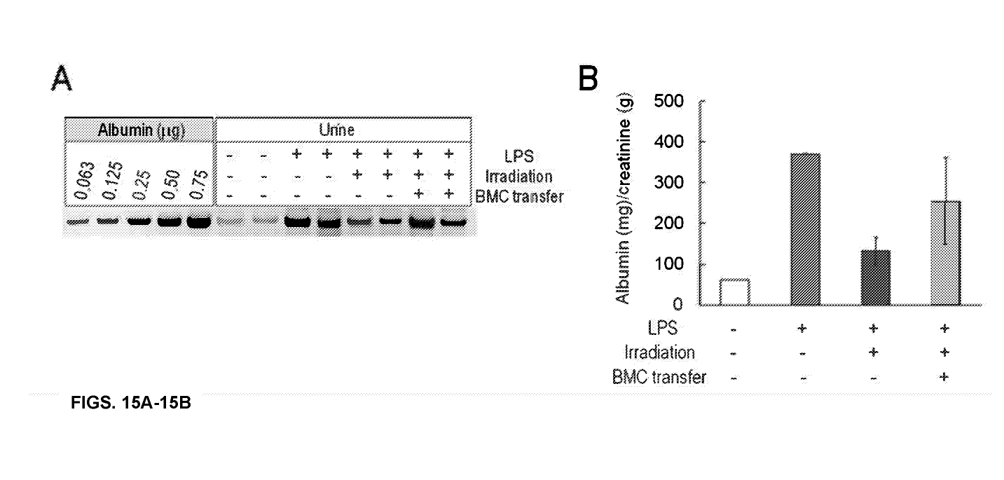

To further test our hypothesis that BM myeloid cells are required for suPAR production and proteinuria development, we reasoned that myeloablation via irradiation could prevent suPAR-mediated proteinuria following LPS treatment. BALB/c mice were irradiated prior to LPS injection (FIG. 9H). The irradiation led to a significant reduction in the degree of proteinuria (FIG. 9I, and FIGS. 15, A and B) as well as plasma suPAR levels (FIG. 9J). However, transfer of normal BM cells into recipient mice following irradiation, but prior to LPS-injection, again resulted in proteinuria with elevated plasma suPAR levels (FIGS. 9, I and J). Together, these results indicate that BM myeloid cells are necessary for LPS-induced proteinuria and associated increases in plasma suPAR.

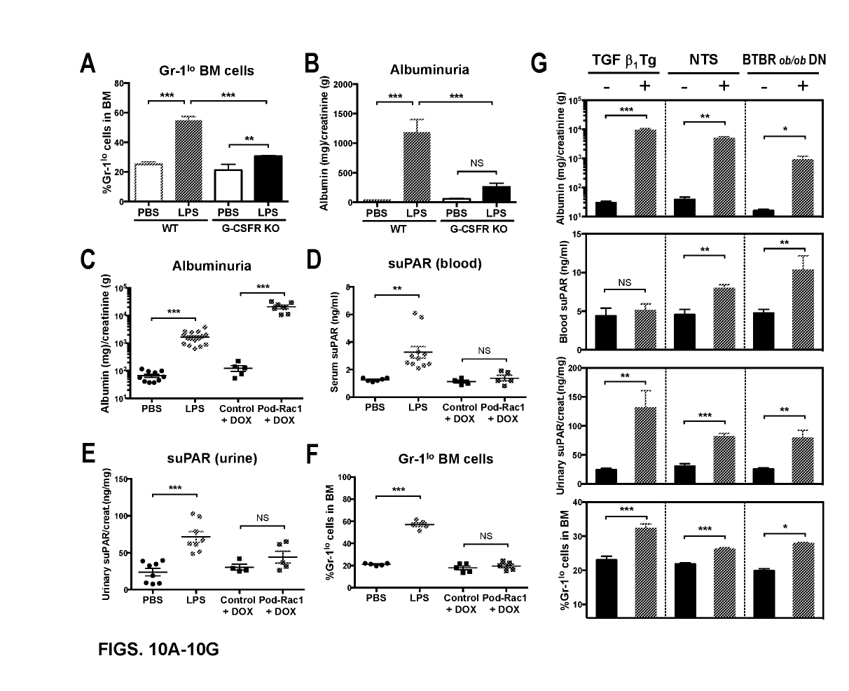

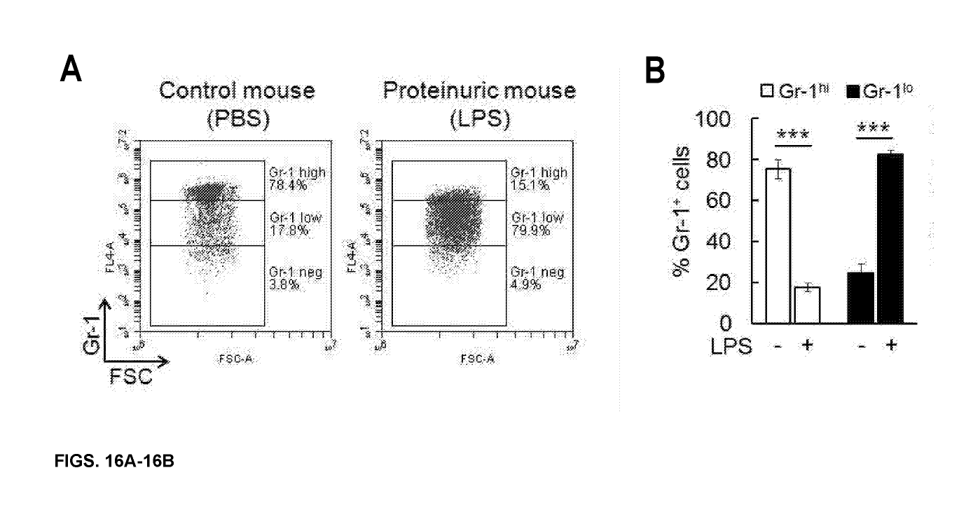

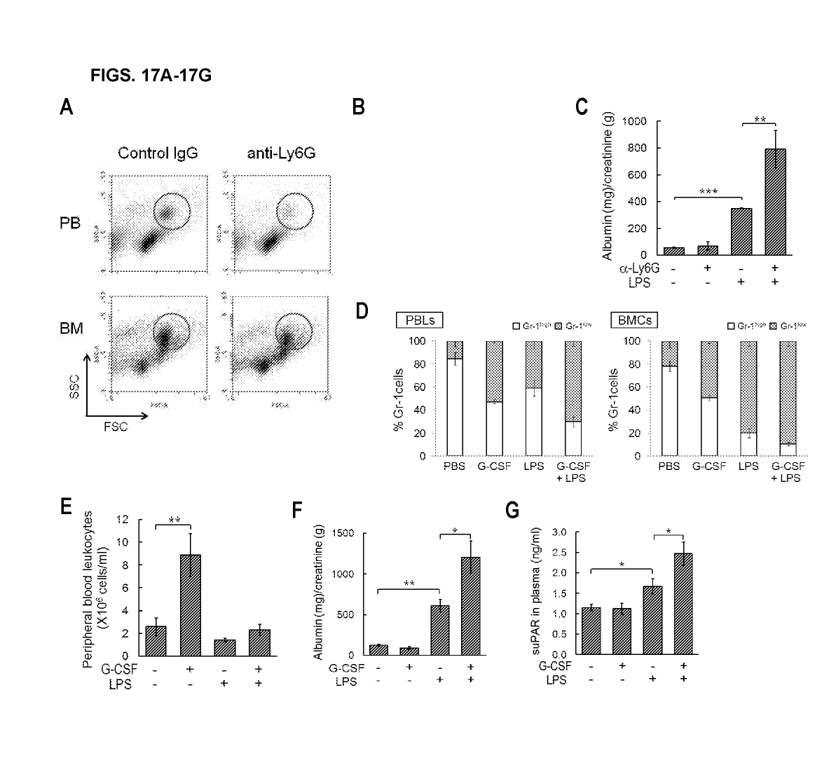

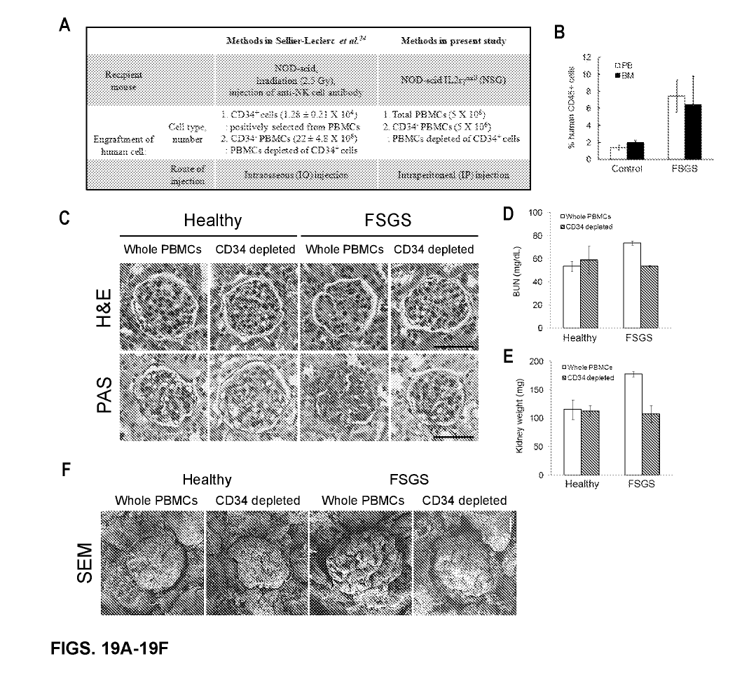

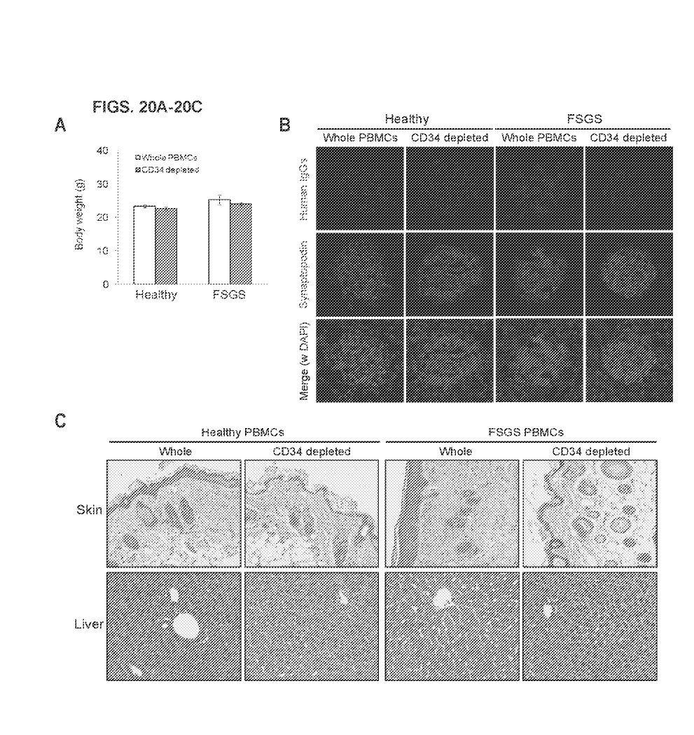

To define the nature of the BM myeloid cells, we studied the expression of granulocyte differentiation antigen, Gr-1. The surface expression of Gr-1 is representative of the maturation status of myeloid cells (Basu et al., 2002). In the BM, the level of Gr-1 expression is low on myeloid progenitor or immature cells and increases as they mature to granulocytes. In WT mice, LPS stimulation led to a significant increase in the percentage of Gr-1^{lo} cells in the BM (FIG. 19A, and FIGS. 16, A and B). However, loss of granulocyte colony-stimulating factor (G-CSF) receptor (G-CSFR), a major regulator of myelopoiesis (Liu et al., 1996), resulted in impaired expansion of Gr-1^{lo} BM myeloid cells upon LPS treatment (FIG. 10A), and accordingly, reduced level of proteinuria (FIG. 10B). We therefore hypothesized that the reactive expansion of immature myeloid cell populations in BM may contribute to the pathogenic kidney process. We tested this with eradication of peripheral mature neutrophils in vivo using anti-Ly6G antibody and observed increased proteinuria in LPS-challenged mice (FIG. 17, A-C). Similarly, treatment with G-CSF to stimulate myelopoiesis (FIGS. 17, D and E), accelerated proteinuria and increased plasma suPAR levels compared to LPS alone (FIGS. 17, F and G).

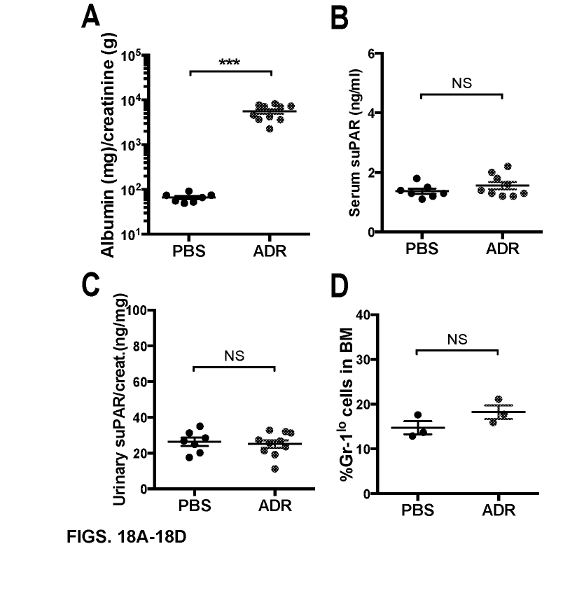

Given recent data on suPAR as a risk factor in CKD (Hayek et al., 2015), we reasoned that the expansion of Gr-1^{lo} BM myeloid cells could be a common feature of suPAR-associated proteinuria. Thus, we examined the levels of suPAR and Gr-1^{lo} BM myeloid cells in 5 different animal models of proteinuria. i) A genetic model of podocyte injury, in which constitutively active mutant of Rac1 is expressed from the podocyte-specific promoter (NEF-rtTA×Rac1) with doxycycline (DOX) diet (thereafter referred as Pod-Rac1) (Yu et al., 2013), ii) Adriamycin (ADR)-induced nephropathy, a cytotoxin-mediated podocyte mitochondrial injury model (Papeta et al., 2010), iii) Albumin TGF β₁ transgenic (TGF β₁ Tg) mouse model that develops severe fibrosing kidney disease (Kopp et al., 1996, Schiffer et al., 2001), iv) Nephrotoxic serum (NTS) nephritis, a rodent model of glomerulonephritis (GN) (Kistler et al., 2013), and v) BTBR ob/ob mice, a mouse model of diabetic nephropathy (DN) (Hudkins et al., 2010). All tested animals exhibited proteinuria after their respective induction (FIGS. 10, C and G, FIG. 18A). Unlike LPS model, Pod-Rac1 and ADR models, in which podocytes are the direct target of injury, we did not find elevated suPAR levels in the circulation or urine (FIGS. 10, D and E, FIGS. 18, B and C). There was also no significant increase of the percentage of Gr-1^{lo} myeloid cells in BM (FIG. 10F, FIG. 18D). In contrast, TGF β₁ Tg, NTS, and BTBR ob/ob mice, showed elevated suPAR levels, which was accompanied by an expansion in Gr-1^{lo} BM myeloid cells (FIG. 10G) as seen in the LPS model. Taken together, these results suggest that expansion of Gr-1^{lo} BM myeloid cells could be a common upstream event in immunologically associated forms of proteinuria, that results in elevated systemic suPAR, podocyte injury and the development of proteinuria.

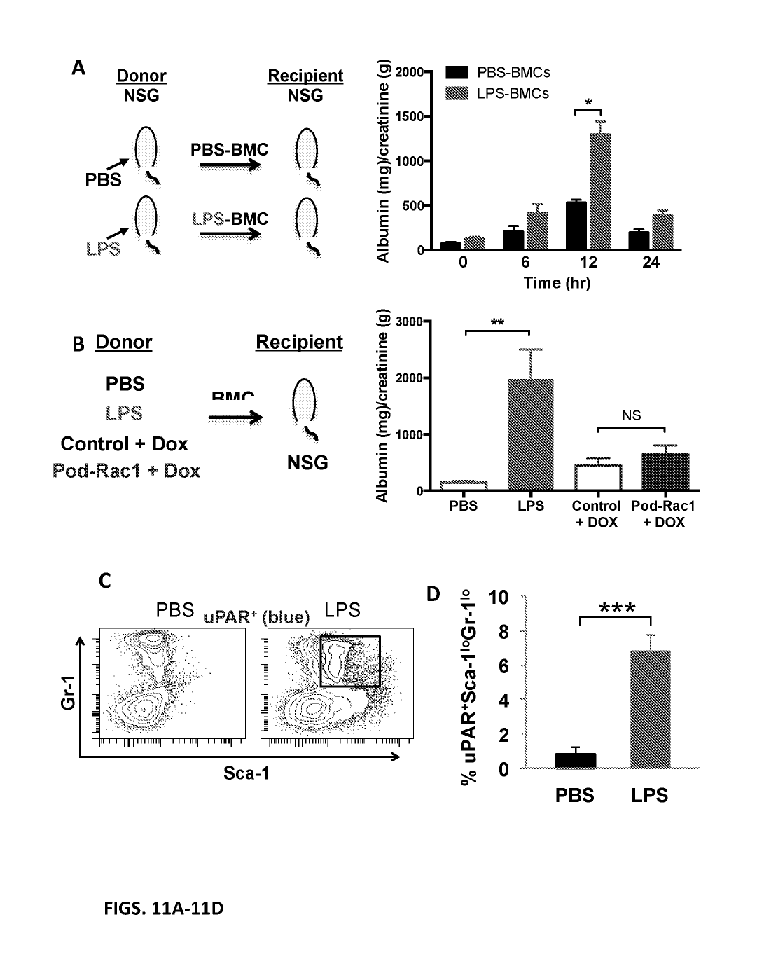

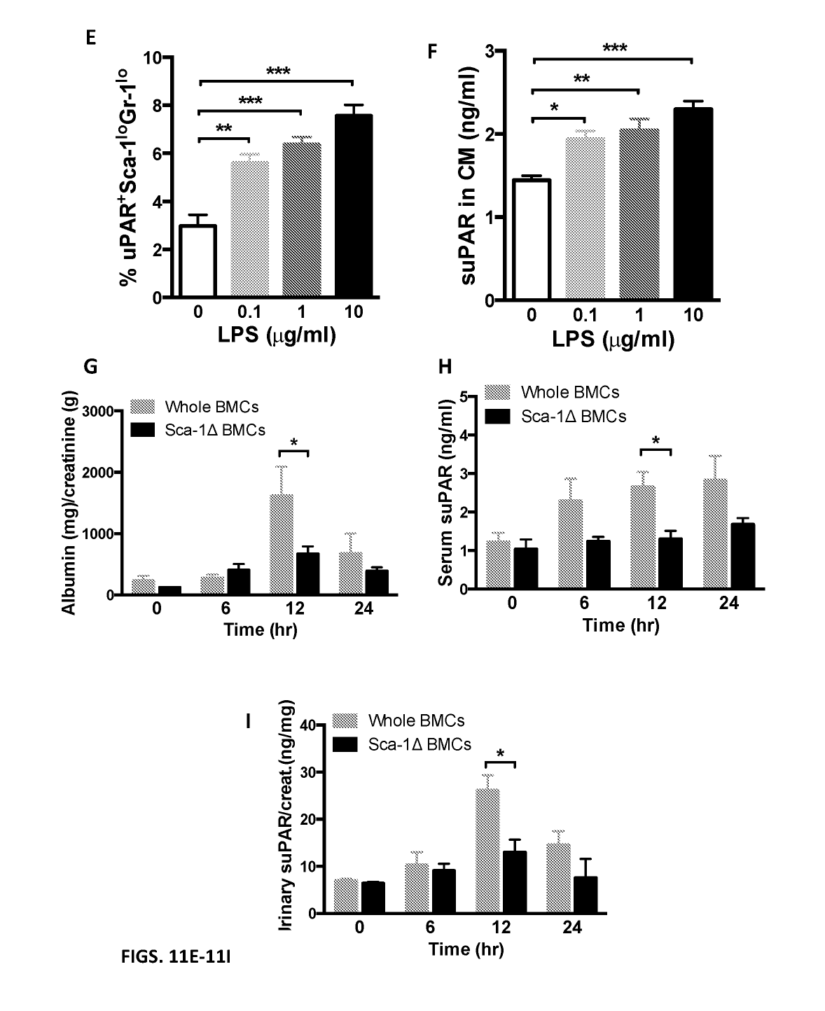

Next, we evaluated whether Gr-1^{lo} BM myeloid cells harvested from proteinuric mice were able to induce proteinuria when injected into healthy animals (FIGS. 11, A and B). To test this, BM (primarily myeloid) cells isolated from PBS- or LPS-injected NSG mice were adoptively transferred into unchallenged NSG recipient mice. Indeed, transfer of LPS-challenged BM cells induced proteinuria in healthy recipient mice (FIG. 11A). Similarly, LPS-challenged WT C57BL6 mouse BM cells were able to induce proteinuria in recipient mice. However, the BM cells of Pod-Rac1 proteinuric mice did not cause proteinuria (FIG. 11B) suggesting that podocyte injury per se does not induce Gr-1^{lo} BM myeloid cells to become kidney pathogenic; this interpretation is consistent with low systemic suPAR levels and low percentage of Gr-1^{lo} myeloid cells in BM of Pod-Rac1 proteinuric mice.

Since Gr-1^{lo} myeloid cells in the BM are heterogeneous, we sought to determine which subsets of uPAR-expressing Gr-1^{lo} cells are responsible for development of proteinuria. Given that stem cell antigen-1 (Sca-1) is expressed on mouse hematopoietic stem/progenitor cell (HSPC) populations among other cell types and is highly responsive to proliferative stimuli (Holmes et al., 2007), we examined Sca-1 expression in uPAR-expressing Gr-1^{lo} BM cells. LPS stimulation increased uPAR-expressing BM cell population (shown in blue in FIG. 11C) that is Sca-1^{lo} Gr-1^{lo}, which suggests uPAR-expressing cells are myeloid lineage-committed progenitor cells (defining myeloid progenitor cells, MPCs) (FIGS. 11, C and D).

To test whether the uPAR-expressing MPCs are capable of secreting suPAR, we studied suPAR secretion using in vitro BM cell culture under LPS stimulation. Consistent with in vivo studies, LPS stimulated the expansion of uPAR-expressing MPCs (FIG. 11E) as well as suPAR secretion (FIG. 11F) into the culture medium in a dose-dependent manner. To further evaluate if the MPCs can transfer disease when injected into healthy mice, Sca-1⁺ cells were depleted from LPS-challenged BM cells by magnetic separation prior to transfer into NSG mice (FIG. 7B). The removal of Sca-1⁺ BM cells lowered both proteinuria (FIG. 11G) and suPAR levels (FIGS. 11, H and I), compared to non-depleted control BM cells. These results emphasize the primary role of BM MPCs in regulation of suPAR production and proteinuria development.

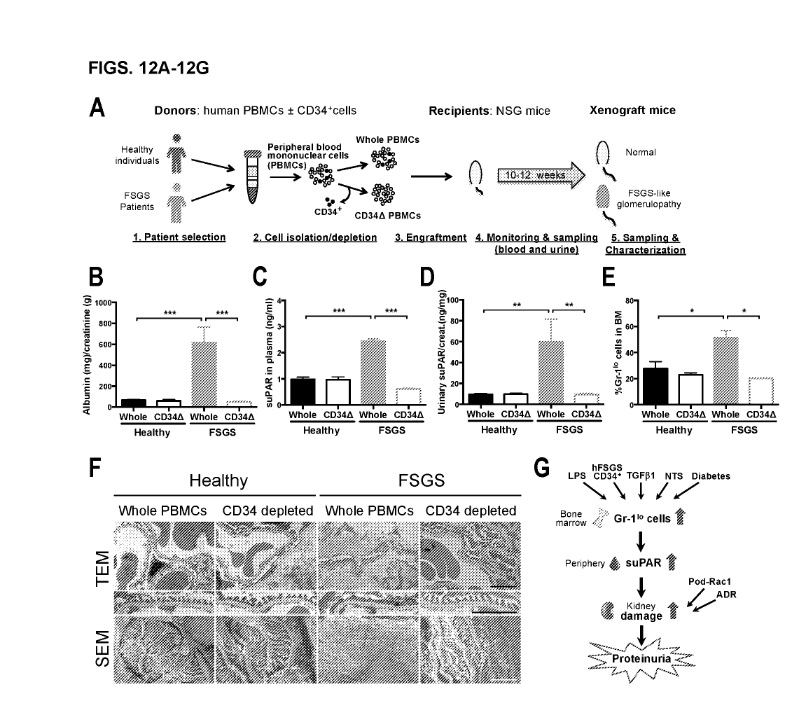

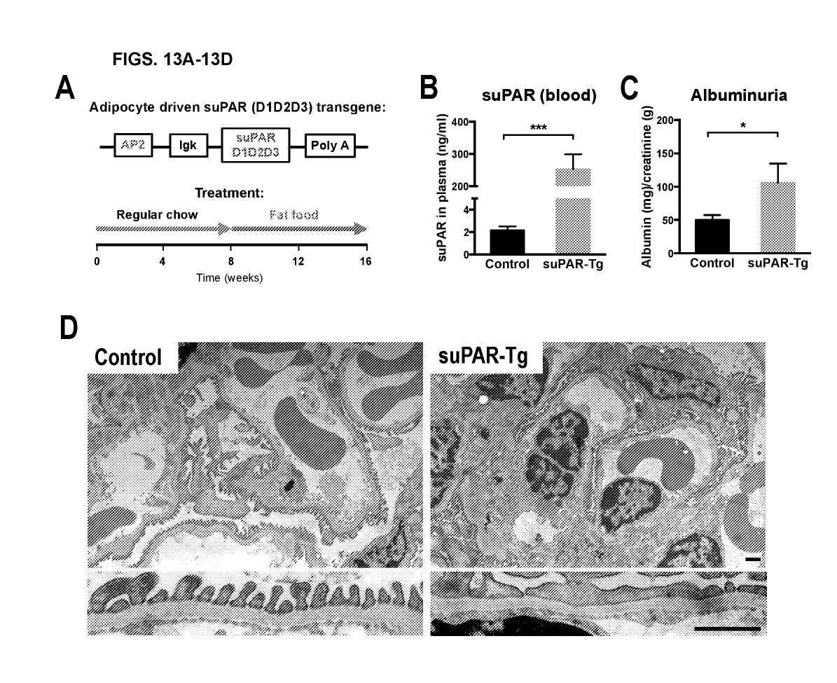

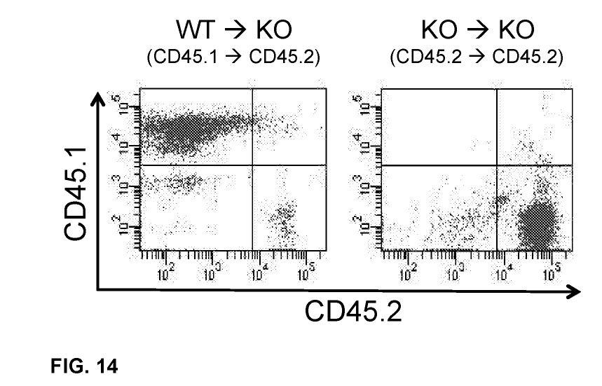

In an effort to translate our findings into humans, we utilized a humanized mouse approach (Schultz et al., Brehm et al, Ito et al.). A common model used is the immunodeficient mouse engrafted with human hematopoietic cells, such as peripheral blood mononuclear cells (PBMCs) or hematopoietic stem/progenitor cells (HSPCs). Sellier-Leclerc and colleagues described that transfer of CD34⁺ HSPCs from patients with glomerular diseases into healthy mice can induce albuminuria, but offered no mechanism for their findings (Sellier-Leclerc et al., 2007). We hypothesized that human CD34⁺ cells isolated from patients with recurrent FSGS (and high suPAR levels) might induce proteinuria via suPAR-driven podocyte injury. To test this hypothesis, using a modified method (FIG. 19A), we introduced whole PBMCs or PBMCs depleted of CD34⁺ cells (CD34Δ PBMCs) derived from FSGS patients or healthy individuals into the NSG mice (FIG. 12A). Engraftment rates of human cells were determined by the percentage of human CD45⁺ cells in blood and bone marrow (BM) of the recipient animals (FIG. 19B). Notably, the mice receiving patient whole PBMCs, but not CD34Δ PBMCs, developed proteinuria (FIG. 4B) in association with high mouse suPAR levels in both blood and urine (FIGS. 12, C and D). These mice had also an increased percentage of Gr-1^{lo} population in BM (FIG. 12E) similar to what we have observed in the suPAR-associated proteinuric animal models. This disease phenotype was prevented by depletion of CD34+ cells from the patients (FIG. 12E). Proteinuria in mice receiving patient whole PBMCs was accompanied by mild glomerular sclerosis (FIG. 19C) and extensive podocyte foot process effacement (FIG. 12F). These mice also exhibited elevated blood urea nitrogen (BUN) levels, increased kidney weight, and enlarged, loosely structured glomeruli (FIG. 19, D-F). It is unlikely that the observed impairment in the kidney function was due to the graft-versus-host disease (GVHD) since PBMCs from the healthy individuals as well as those depleted of CD34⁺ cells derived from FSGS patients did not cause kidney injury. The xenograft mice did not exhibit body weight loss either (FIG. 20A). Moreover, we only observed negligible glomerular deposit of human IgGs (FIG. 20B) as well as very mild lymphocyte infiltration in skin and liver (FIG. 20C) of the xenograft mice. Together, these data suggest that engraftment of CD34⁺ cells derived from patients with recurrent FSGS into NSG mice caused expansion of Gr-1^{lo} BM myeloid cells, leading to suPAR-driven podocyte injury that results in proteinuria.

In summary, our study suggests that proteinuric diseases accompanied by high suPAR may originate as a primary BM progenitor cell disorder. Unlike local podocyte injury models (e.g. genetic mutations in podocyte-specific genes), the systemic immunological models (LPS, TGF β₁ Tg, NTS, and BTBR ob/ob mouse models) and the xenograft mouse model of human FSGS converge at the expansion of Gr-1^{lo} cells in BM and high suPAR levels. The expansion of Gr-1^{lo} BM cells could be a common upstream event that leads to suPAR-driven podocyte injury in the development of proteinuria and possibly CKD. In addition, our findings also serve as a prototype for the pathogenesis of other diseases in which BM progenitors may regulate organ function via soluble mediators presently categorized as ‘idiopathic’ yet may rather evolve through similar or different mediators and diverge from the classical inflammatory response.

Methods

Human subjects. Peripheral blood was drawn from healthy volunteers and patients with FSGS and in accordance with guidelines on human research and approval of the Institutional Review Board of Rush University Medical Center. Informed consent was obtained from the blood donors.

Mice. 10-12-week-old BALB/c, C57BL/6, Plaur^{-/-} (uPAR KO), B6.SJL (CD45.1), csf3r^{-/-} (G-CSFR null), BTBR/ob heterozygotes (BTBRob^{+/-}; BTBR.V(B6)-Lep^{ob}/WiscJ), and NOD-scid IL2rγ^{null} (NSG), complement receptor 3 (CR3) null mice were used (from Jackson laboratory, USA). The Plaur^{-/-} mice were originally on a mixed background of 75% C57BL/6 and 25% 129, but backcrossed to C57BL/6 mice for more than ten generations before any use. EGFP_CA-Rac1 transgenic, and Nphs1-rtTA (NEF-rtTA) mice were kindly provided by Andrey S. Shaw (Washington University School of Medicine, Missouri). (Yu et al.) Albumin TGF β1 transgenic and littermate mice were kindly provided by Markus Bitzer (University of Michigan). (Kopp et al.) Animal experiments were carried out according to the National Institutes of Health Guide for Care and Use of Experimental Animals and approved by the Rush University Institutional Animal Care and Use Committee (IACUC).

Albumin creatinine ratio (ACR) measurement Mouse urine samples were collected and urinary albumin and creatinine were measured by mouse albumin ELISA (Bethyl Labs), and creatinine assay (Cayman chemical) kits according to manufacturers' protocols. The ratio of urinary albumin to creatinine was then calculated.

Detection of circulating and urinary suPAR levels Mouse suPAR levels from plasma (and/or serum) and urine were evaluated by enzyme-linked immunosorbent assay (ELISA) kit (R&D systems) following the manufacturer's protocol.

Generation of suPAR transgenic mice. suPAR transgenic mouse (suPAR-Tg) model was created that drives mouse full-length suPAR (corresponding to NP_035243, DIDIIDIII without GPI anchor) expression from adipocytes with consequent release into circulation. To stimulate suPAR production, regular rodent diet was replaced by high fat food when the mice were at least 2 months old. Two months after switching to high fat diet.

Generation of BM chimeric mice. uPAR deficient mice (uPAR KO, CD45.2) were irradiated with a dose of 9.5 Gy and reconstituted via retro-orbital injection with 1×10⁷ BM cells of either uPAR WT (B6.SJL, CD45.1) or uPAR KO (CD45.2) mice. Mice were administered antibiotic-treated water and used for experiments at 6 weeks after BM transplantation. Engraftment of the donor cells was evaluated by flow cytometric analysis of peripheral blood leukocytes stained with fluorescence-conjugated antibodies against CD45.1 (uPAR WT) and CD45.2 (uPAR KO).

Engraftment of human PBMCs into NSG mice. PBMCs were isolated from peripheral blood of healthy volunteers and patients with recurrent FSGS by standard ficoll separation. The purified PBMCs were transferred via intraperitoneal (i.p) injection into NSG mice (5×10⁶ cells per mouse) on day 0. The engrafted mice were monitored over time for the development of FSGS-like phenotypes by monitoring in the blood and urine; proteinuria and high suPAR levels. Freshly isolated PBMCs were utilized for transfer as the freeze-thawed PBMCs showed poor engraftment in our experimental settings. For measurement of reconstitution rates of human cells, peripheral blood samples were drawn from the mice 10˜12 weeks post-engraftment and blood leukocytes were stained with fluorescence-labeled antibodies against human CD45 and mouse CD45. Following RBC lysis, the cells were analyzed by flow cytometry.

Proteinuric animal models. i) LPS injected mice. Proteinuria was induced with single injection of LPS as described previously (Wei et al., 2008; Wei et al.,). Briefly, mice were injected with LPS (Sigma) i.p at a dose of 10 mg per kg body weight. The mice injected with PBS were used as a control. Twenty-four hours after LPS treatment, urine and blood samples were collected and kidney, femurs, and tibias were harvested from the sacrificed mice.

ii) Pod-Rac1, Inducible EGFP_CA-Rac1 transgenic mice. EGFP_CA-Rac1 transgenic mice were crossed to Nphs1-rtTA (NEF-rtTA) inducer mice to generate double transgenic (dTG, NEF×Rac1) mice. Male mice were used in this study. To induce transgene expression, regular chow was replaced with doxycycline (DOX)-supplemented chow (2,000 ppm; TestDiet). Single transgenic (EGFP_CA-Rac1) mice were used as a control. Twelve days after DOX diet, urine and blood samples were collected and kidney, femurs, and tibias were harvested from the sacrificed mice. Hi) Adiamycin (ADR) injected mice: Proteinuria was induced with single injection of ADR as described previously. (Wang et al.) Briefly, male BALB/c mice were injected with ADR (Doxorubicin Hydrochloride, Sigma) via the tail vein at a dose of 11 mg per kg body weight. Six days after ADR injection, urine and blood samples were collected and kidney, femurs, and tibias were harvested from the sacrificed mice. iv) TGF β1 transgenic mice: The transgenic mice that express an active form of TGF β1 under the control of murine albumin promoter developed proteinuria as described previously (Kopp et al.). Since the transgene is on the Y chromosome, male mice (<4-week-old) with severe phenotype were used. Littermate female mice were used as a control. v) Nephrotoxic serum (NTS) nephritis model: Proteinuria was induced by injection of NTS as described previously (Kistler et al.). Briefly, male C57BL6 mice were injected i.p with sheep anti-rabbit glomerular basement membrane antibody at a dose of 500 mg per kg body weight on consecutive days. The mice injected PBS were used as a control. Seven days after injection, urine and blood samples were collected and kidney, femurs, and tibias were harvested from the sacrificed mice. vi) BTBR ob/ob diabetic nephropathy (DN) model: The mouse strain BTBR with the ob/ob leptin-deficiency mutation developed severe type 2 diabetes with heavy proteinuria as described previously (Hudkins et al.). Wild-type (BTBR ob^{+/+}), heterozygous (BTBR ob^{+/-}), and homozygous (BTBR ob^{-/-}) mice were obtained by mating heterozygous (BTBR ob^{+/-}) mice. For DN model, 16-week-old homozygotes (BTBR ob^{-/-}) were used. Wild-type (BTBR ob^{+/+}) or heterozygote (BTBR ob^{+/-}) littermates were used as controls.

Purification of mouse PBLs and BMCs. To collect blood, mice were anesthetized, then the blood was drawn from posterior vena cava into acid-citrate-dextrose (ACD, Sigma) solution-containing 1 ml syringe. After lysis of red blood cells (RBCs), blood leukocytes were washed and counted. For BMCs purification, femurs and tibias were taken and flushed with a syringe filled with Hank's balanced salt solution (HBSS, Life Technologies) containing 0.1% bovine serum albumin and 20 mM HEPES (pH 7.4). Following RBC lysis, the BM cell suspensions were filtered through a 40 μm cell strainer (Falcon).

Adoptive transfer of BMCs. To explore the role of BMCs in production of suPAR in proteinuric mice, BALB/c mice were lethally irradiated with 9.5 Gy using Gammacell 40. On the next day, these mice were received syngeneic donor BMCs, which were labeled with Calcein-AM (Life Technologies), via the retro-orbital route (5×10⁶ cells per mouse). To induce proteinuria, LPS was injected into the recipient mice 1 hour after transfer of BMCs. Twenty-four hours after LPS treatment, urine and blood samples were collected and kidney, femurs, and tibias were harvested from the sacrificed mice. To better understand the role of BM-derived suPAR in development of proteinuria, donor BMCs were harvested from WT (C57BL/6) or Plaur^{-/-} mice and transferred into lethally irradiated WT mice (12 Gy). Twenty-four hours following LPS challenge, ACR and suPAR levels were measured using the collected urine and blood samples.

BM cell transfer from proteinuric animal models into NSG mice. To test the ability of BMCs on development of proteinuria directly, donor (WT or Plaur^{-/-}) mice were injected with LPS, 24 hours later, BMCs were isolated and transferred into NSG recipient mice. ACR and suPAR levels were monitored in a time course. To determine if Sca-1⁺ cells are required for suPAR-mediated proteinuria, BMCs were isolated from LPS-challenged WT mice and divided into 2 groups, whole BMCs and Sca-1⁺ cell depleted BMCs. And these cells were transferred into NSG mice. ACR and suPAR levels were monitored in a time course.

To evaluate if Gr-1^{lo} BM myeloid cells from mice with proteinuria are able to induce proteinuria, the BM cells were isolated from different proteinuric animal models, then transferred into NSG mice. Twelve hours following transfer of donor BM cells, ACR was measured from the urine samples of the recipient mice. To determine if Sca-1⁺ cells are required for suPAR-mediated proteinuria, BM cells were isolated from LPS-challenged WT mice and divided into 2 groups, whole BM cells and Sca-1⁺ cell depleted BM cells and these cells were transferred into NSG mice. ACR and suPAR levels were monitored over time.

In vivo depletion. Anti-Ly6G monoclonal antibody (1A8, BioXcell) or control rat IgG2a antibody (2A3, BioXcell) was injected i.p into mice (500 μg per mouse) 48 hours and 1 hour prior to LPS injection. Neutrophil depletion was confirmed by flow cytometric analysis.

Treatment of granulocyte colony stimulating factor (G-CSF). Recombinant mouse G-CSF (Shenandoah Biotechology INC) was administrated by daily i.p injection into BALB/c mice at a dose of 4 μg per 20 g body weight for 2 consecutive days. Blood cell counts were determined using a Hemavet 950FS (Drew Scientific).

Treatment of plerixafor. Plerixafor (AMD3100, Sigma) was administrated by daily i.p. injection into BALB/c mice at a dose of 100 μg per 20 g body weight for two consecutive days.

Cell depletion methods. To study adoptive transfer of Sca-1+ cell depleted BMCs, Sca-1⁺ cells were removed from whole BMCs using EasySep™ mouse cell isolation kits (Stemcell technologies). In brief, LPS-challenged WT BMCs were isolated and labeled with biotinylated anti-mouse Sca-1 antibody and followed by incubation with streptavidin-magnetic micro beads. By collecting unbound cells in the magnetic field, Sca-1⁺ cell-depleted BMCs were prepared. For the humanization study, human CD34⁺ cells were depleted from PBMCs obtained from patients with FSGS and healthy donors, using EasySep™ human CD34 positive selection kit (Stemcell technologies).

Flow cytometric analysis. Isolated PBLs or BMCs were resuspended with phosphate buffered saline (PBS) and single cell suspensions were first incubated with rat anti-mouse CD16/CD32 (BD biosciences) for 30 minutes on ice to block Fc receptors. And the cells were labeled for 30 minutes on ice with fluorescence-conjugated antibodies: Gr-1-allophycocyanin (APC), uPAR-phycoerythrin (PE), Sca-1-fluorescein isothiocyanate (FITC), c-kit-APC, and irrelevant IgG isotype control antibodies. Data acquisition and analysis were performed on an Accuri C6 flow cytometer (BD biosciences).

in vitro LPS stimulation on isolated BM cells. The BM cells were isolated from C57BL6 WT mice and treated with PBS or various concentrations of LPS (0.1, 1, and 10 μg/ml). Following 24-hour-incubation at 37 C in a CO2 incubator (5%), the cells and culture supernatants were collected. The cells were stained for uPAR, Sca-1, and Gr-1. The in vitro induction of uPAR+Sca-1^{lo}Gr-1^{lo} cells in total BM cells was determined by triple color-flow cytometric analysis. The culture supernatants were used for suPAR measurement.

Histology. Kidneys were dissected from the mice. The renal tissues were fixed overnight in formalin, and embedded in paraffin. The sections were cut at 3-4 μm thickness, and stained with hematoxylin and eosin (H&E) and Periodic acid-Schiff (PAS).

Electron microscopy. Kidneys were dissected from the humanized mice. Renal tissues were cut down to 2-3 mm pieces. For SEM, tissues were fixed in Trumps Fixative (EMS, cat. #11750), dehydrated with graded ethanol, dried using the 850 Critical Point Dryer (EMS) and gold coated on the 108 Auto Sputter Coater (Cressington). For TEM, renal tissues were fixed as before and post fixed in 1% OsO4 for 1 hour. Tissues were washed, dehydrated and embedded in Epon812. Ultrathin kidney sections (70 nm) obtained on the EM UC7 Ultramicrotome (Leica) were placed on Formvar coated Ni slot grids (EMS, cat. # FF-2010-Ni) and stained in 5% uranyl acetate and 0.1% lead citrate. EM micrographs were taken on the Sigma HDVP Electron Microscope (Zeiss).

Immunofluorescence. Frozen kidney tissues of the xenograft mice were cut at 4 μm thickness and fixed with ice-cold acetone for 5 minutes. After blocking with blocking solution (2.5% donkey normal serum and 2.5% fetal bovine serum (FBS) in PBS) for 1 hour, samples were stained with goat anti-mouse synaptopodin (sc-21537, Santa Cruz Biotechnology) diluted at 1:300 with blocking solution, and followed by Alexa Fluor 546-conjugated donkey anti-goat IgG (Molecular Probes) diluted at 1:1000 with blocking solution. After washing, the samples further incubated with Alexa Fluor 488-conjugated goat anti-human IgG (109-545-006, Jackson ImmunoResearch) diluted at 1:300 with blocking solution. After washing, stained samples were mounted with ProLong® Gold antifade reagent with DAPI (Molecular Probes). Images were obtained and analyzed by using a Carl Zeiss LSM 700 confocal microscopy.

Statistical analysis. Data are shown as mean±SD or ±SEM. Statistical analysis was assessed by Graph Pad Prism 5.0 (*p<0.05, **p<0.01, ***p<0.001). Significance of the difference between two groups was assessed using Student's unpaired two-tailed t test. For multiple group comparisons, we performed one-way ANOVA with Turkey's multiple comparison test.

The above Figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

### REFERENCES

1. M. P. Winn et al., Science 308, 1801 (Jun. 17, 2005).
2. V. D. D'Agati, F. J. Kaskel, R. J. Falk, N Engl J Med 365, 2398 (December 22).
3. P. Cravedi, J. B. Kopp, G. Remuzzi, Am J Transplant 13, 266 (February).
4. A. B. Fogo, Mechanisms of progression of chronic kidney disease. Pediatr Nephrol 22, 2011-2022 (2007).
5. L. Gallon, J. Leventhal, A. Skaro, Y. Kanwar, A. Alvarado, N Engl J Med 366, 1648 (April 26).
6. R. J. Shalhoub, Lancet 2, 556 (Sep. 7, 1974).
7. C. Wei et al., Modification of kidney barrier function by the urokinase receptor. Nat Med 14, 55-63 (2008).
8. C. Wei et al., Circulating urokinase receptor as a cause of focal segmental glomerulosclerosis. Nat Med 17, 952-960.
9. M. Thuno, B. Macho, J. Eugen-Olsen, Dis Markers 27, 157-172 (2009).
10. F. Blasi, P. Carmeliet, Nat Rev Mol Cell Biol 3, 932-943 (December, 2002).
11. H. W. Smith, C. J. Marshall, Nat Rev Mol Cell Biol 11, 23-36 (January).
12. B. K. Pliyev, Mol Cell Biochem 321, 111 (January, 2009).
13. B. K. Pliyev, M. Y. Menshikov, Inflammation 33, 1 (February).
14. P. E. Dekkers, T. ten Hove, A. A. to Velde, S. J. van Deventer, T. van Der Poll, Infect Immun 68, 2156 (April, 2000).
15. M. Tjwa et al., J Clin Invest 119, 1008 (April, 2009).
16. L. D. Shultz et al., J Immunol 174, 6477 (May 15, 2005).
17. R. Duran-Struuck, R. C. Dysko, J Am Assoc Lab Anim Sci 48, 11 (January, 2009).
18. S. Basu, G. Hodgson, M. Katz, A. R. Dunn, Blood 100, 854-861 (Aug. 1, 2002).
19. H. Yu et al., Rac1 activation in podocytes induces rapid foot process effacement and proteinuria. Mol Cell Biol 33, 4755-4764 (2013).
20. A. L. Sellier-Leclerc et al., A humanized mouse model of idiopathic nephrotic syndrome suggests a pathogenic role for immature cells. J Am Soc Nephrol 18, 2732-2739 (2007)
21. M. Nishimura et al., J Exp Med 179, 1053 (Mar. 1, 1994).
22. S. S. Hayek et al., Soluble Urokinase Receptor and Chronic Kidney Disease. N Engl J Med 373, 1916-1925 (2015).
23. D. Cathelin et al., Administration of recombinant soluble urokinase receptor per se is not sufficient to induce podocyte alterations and proteinuria in mice. J Am Soc Nephrol 25, 1662-1668 (2014).
24. J. M. Spinale et al., A reassessment of soluble urokinase-type plasminogen activator receptor in glomerular disease. Kidney Int 87, 564-574 (2015).
25. M. Alfano et al., Full-length soluble urokinase plasminogen activator receptor down-modulates nephrin expression in podocytes. Sci Rep 5, 13647 (2015).
26. L. D. Shultz, M. A. Brehm, J. V. Garcia-Martinez, D. L. Greiner, Humanized mice for immune system investigation: progress, promise and challenges. Nat Rev Immunol 12, 786-798.
27. F. Liu, H. Y. Wu, R. Wesselschmidt, T. Kornaga, D. C. Link, Impaired production and increased apoptosis of neutrophils in granulocyte colony-stimulating factor receptor-deficient mice. Immunity 5, 491-501 (1996).
28. N. Papeta et al., Prkdc participates in mitochondrial genome maintenance and prevents Adriamycin-induced nephropathy in mice. J Clin Invest 120, 4055-4064 (2010).
29. J. B. Kopp et al., Transgenic mice with increased plasma levels of TGF-beta 1 develop progressive renal disease. Lab Invest 74, 991-1003 (1996).
30. M. Schiffer et al., Apoptosis in podocytes induced by TGF-beta and Smad7. J Clin Invest 108, 807-816 (2001).
31. A. D. Kistler et al., Transient receptor potential channel 6 (TRPC6) protects podocytes during complement-mediated glomerular disease. J Biol Chem 288, 36598-36609 (2013).
32. K. L. Hudkins et al., BTBR Ob/Ob mutant mice model progressive diabetic nephropathy. J Am Soc Nephrol 21, 1533-1542 (2010).
33. C. Holmes, W. L. Stanford, Concise review: stem cell antigen-1: expression, function, and enigma. Stem Cells 25, 1339-1347 (2007).
34. M. A. Brehm, L. D. Shultz, D. L. Greiner, Humanized mouse models to study human diseases. Curr Opin Endocrinol Diabetes Obes 17, 120-125.
35. R. Ito, T. Takahashi, I. Katano, M. Ito, Current advances in humanized mouse models. Cell Mol Immunol 9, 208-214.
36. Y. Wang, Y. P. Wang, Y. C. Tay, D. C. Harris, Progressive adriamycin nephropathy in mice: sequence of histologic and immunohistochemical events. Kidney Int 58, 1797-1804 (2000).

1. A method of treating kidney diseases or disorders, comprising administering to a subject in need thereof, an effective amount of an agent which inhibits myeloid progenitor cells in the subject from producing soluble urokinase receptor (suPAR).

2. The method according to claim 1, wherein the agent removes CD34+ cells from the subject.

3. The method according to claim 1, wherein the kidney disease or disorder comprises proteinuria.

4. The method according to claim 1, wherein the kidney disease or disorder comprises focal segmental glomerulosclerosis (FSGS).

5. The method according to claim 1, further comprising isolating the myeloid progenitor cells from the subject, transferring the myeloid progenitor cells to a murine host, measuring a number of Gr-1^{lo} cells in the murine host relative to a control number of Gr-1^{lo} cells in a murine control and administering the agent to subjects whose myeloid progenitor cells give rise to an increased number of Gr-1^{lo} cells in the murine host relative to the murine control.

6. The method according to claim 5, wherein the myeloid progenitor cells comprise peripheral blood mononuclear cells (PBMC).

7. The method according to claim 5, wherein the myeloid progenitor cells are enriched for CD34+ cells.

8. The method according to claim 1, comprising removing CD34+ cells from the subject having an increased number of Gr-1^{lo} cells in the murine host relative to the murine control.

9. The method according to claim 1, wherein the agent comprises an antibody, aptamer, antisense oligonucleotide, a natural agent, a synthetic agent or combinations thereof.

10. The method according to claim 1, wherein the kidney disease or disorder comprises: podocyte diseases or disorders, proteinuria, glomerular diseases, membranous glomerulonephritis, focal segmental glomerulonephritis, minimal change disease, nephrotic syndromes, pre-eclampsia, eclampsia, kidney lesions, collagen vascular diseases, stress, strenuous exercise, benign orthostatic (postural) proteinuria, focal segmental glomerulosclerosis (FSGS), IgA nephropathy, IgM nephropathy,
membranoproliferative glomerulonephritis, membranous nephropathy,
sarcoidosis, Alport's syndrome, diabetes mellitus, kidney damage due to drugs, Fabry's disease, infections, aminoaciduria, Fanconi syndrome, hypertensive nephrosclerosis, interstitial nephritis, Sickle cell disease, hemoglobinuria, multiple myeloma, myoglobinuria, diabetic nephropathy (DN), lupus nephritis, Wegener's Granulomatosis or Glycogen Storage Disease Type 1.

11. A method of identifying an agent for treatment of kidney diseases or disorders, the method comprising:
administering the agent to a murine host, the murine host having an increased number of Gr-1^{lo} cells relative to a control;
determining the effect of the agent on an indicator of kidney disease or disorder; and
identifying the agent as useful in treating kidney disease or disorder when the agent ameliorates the indicator of kidney disease in the murine host relative to the control.

12. The method according to claim 11, wherein the indicator of kidney disease or disorder is an albumin-to-creatinine ratio (ACR).

13. The method according to claim 11, wherein the indicator of kidney disease or disorder is an amount of suPAR.

14. The method according to claim 11, wherein the indicator of kidney disease or disorder is a proportion of Gr-1^{high} to Gr-1^{lo} cells.

15. The method according to claim 11, wherein the indicator of kidney disease or disorder is a uPAR expression on myeloid cells.

16. The method according to claim 11, wherein the indicator of kidney disease or disorder is podocyte foot process effacement.

17. The method according to claim 11, wherein the agent decreases suPAR activity or expression in the murine host relative to pre-administration activity or expression.

18. The method according to claim 11, wherein the agent inhibits myeloid progenitor cells from producing suPAR.

19. The method according to claim 11, comprising administering myeloid progenitor cells from a subject having FSGS to the murine host to increase the number of Gr-1^{lo} cells relative to a control.

20. The method according to claim 19, comprising enriching the myeloid progenitor cells for CD34+ cells before administering the myeloid progenitor cells to the murine host.
